SEQUENCE LISTING (21 sequences)

SEQ ID NO: 1 - DNA, 4534 bp, Homo sapiens
  CDS (757)..(3774)
gtcacgagcgtcgaagagacaaagccgcgtcagggggcccggccggggcgggggagcccggggcttgttggtgccccagcccgcgcggagggcccttcggacccgcgcgccgccgctgccgccgccgccgcctcgcaacaggtccgggcggcctcgctctccgctcccctcccccgcatccgcgaccctccggggcacctcagctcggccggggccgcagtctggccacccgcttccatgcggttcgggtccaagatgatgccgatgtttcttaccgtgtatctcagtaacaatgagcagcacttcacagaagttccagttactccagaaacaatatgcagagacgtggtggatctgtgcaaagaacccggcgagagtgattgccatttggctgaagtgtggtgtggctctgtagagatagagtttcatcatgttggccaggatggtctcgatctcctgaccttgtgatccgcctgcctcggcctcccaaagtgctggattacaggtgtgagccaccacgatcagcctctagtgtttaaaaaagaacgtccagttgcggataatgagcgaatgtttgatgttcttcaacgatttggaagtcagaggaacgaagttcgcttcttccttcgtcatgaacgcccccctggcagggacattgtgagtggaccaagatctcaggatccaagtttaaaaagaaatggtgtaaaagttcctggtgaatatcgaagaaaggagaacggtgttaatagtcctaggatggatctgactcttgctgaacttcaggaaatggcatctcgccagcagcaacagattgaagcccagcaacaattgctggcaactaaggaacagcgcttaaagtttttgaaacaacaagatcagcgacaacagcaacaagttgctgagcaggagaaacttaaaaggctaaaagaaatagctgagaatcaggaagctaagctaaaaaaagtgagagcacttaaaggccacgtggaacagaagagactaagcaatgggaaacttgtggaggaaattgaacagatgaataatttgttccagcaaaaacagagggagctcgtcctggctgtgtcaaaagtagaagaactgaccaggcagctagagatgctcaagaacggcaggatcgacagccaccatgacaatcagtctgcagtggctgagcttgatcgcctctataaggagctgcagctaagaaacaaattgaatcaagagcagaatgccaagctacaacaacagagggagtgtttgaataagcgtaattcagaagtggcagtcatggataagcgtgttaatgagctgagggaccggctgtggaagaagaaggcagctctacagcaaaaagaaaatctaccagtttcatctgatggaaatcttccccagcaagccgcgtcagccccaagccgtgtggctgcagtaggtccctatatccagtcatctactatgcctcggatgccctcaaggcctgaattgctggtgaagccagccctgccggatggttccttggtcattcaggcttcagaggggccgatgaaaatacagacactgcccaacatgagatctggggctgcttcacaaactaaaggctctaaaatccatccagttggccctgattggagtccttcaaatgcagatcttttcccaagccaaggctctgcttctgtacctcaaagcactgggaatgctctggatcaagttgatgatggagaggttccgctgagggagaaagagaagaaagtgcgtccgttctcaatgtttgatgcagtagaccagtccaatgccccaccttcctttggtactctgaggaagaaccagagcagtgaagatatcttgcgggatgctcaggttgcaaataaaaatgtggctaaagtaccacctcctgttcctacaaaaccaaaacagattaatttgccttattttggacaaactaatcagccaccttcagacattaagccagacggaagttctcagcagttgtcaacagttgttccgtccatgggaactaaaccaaaaccagcagggcagcagccgagagtgctgctatctcccagcataccttcggttggccaagaccagaccctttctccaggttctaagcaagaaagtccacctgctgctgccgtccggccctttactccccagccttccaaagacaccttacttccacccttcagaaaaccccagaccgtggcagcaagttcaatatattccatgtatacgcaacagcaggcgccaggaaaaaacttccagcaggctgtgcagagcgcgttgaccaagactcataccagagggccacacttttcaagtgtatatggtaagcctgtaattgctgctgcccagaatcaacagcagcacccagagaacatttattccaatagccagggcaagcctggcagtccagaacctgaaacagagcctgtttcttcagttcaggagaaccatgaaaacgaaagaattcctcggccactcagcccaactaaattactgcctttcttatctaatccttaccgaaaccagagtgatgctgacctagaagccttacgaaagaaactgtctaacgcaccaaggcctctaaagaaacgtagttctattacagagccagagggtcctaatgggccaaatattcagaagcttttatatcagaggaccaccatagcggccatggagaccatctctgtcccatcatacccatccaagtcagcttctgtgactgccagctcagaaagcccagtagaaatccagaatccatatttacatgtggagcccgaaaaggaggtggtctctctggttcctgaatcattgtccccagaggatgtggggaatgccagtacagagaacagtgacatgccagctccttctccaggccttgattatgagcctgagggagtcccagacaacagcccaaatctccagaataacccagaagaaccaaatccagaggctccacatgtgcttgatgtgtacctggaggagtaccctccatacccacccccaccatacccatctggggagcctgaagggcccggagaagactcggtgagcatgcgcccgcctgaaatcaccgggcaggtctctctgcctcctggtaaaaggacaaacttgcgtaaaactggctcagagcgtatcgctcatggaatgagggtgaaattcaacccccttgctttactgctagattcgtctttggagggagaatttgaccttgtacagagaattatttatgaggttgatgacccaagcctgcccaatgatgaaggcatcacggctcttcacaatgctgtgtgtgcaggccacacagaaatcgttaagttcctggtacagtttggtgtaaatgtaaatgctgctgatagtgatggatggactccattacattgtgctgcctcatgtaacaacgtccaagtgtgtaagtttttggtggagtcaggagccgctgtgtttgccatgacctacagtgacatgcagactgctgcagataagtgcgaggaaatggaggaaggctacactcagtgctcccaatttctttatggagttcaggagaagatgggcataatgaataaaggagtcatttatgcgctttgggattatgaacctcagaatgatgatgagctgcccatgaaagaaggagactgcatgacaatcatccacagggaagacgaagatgaaatcgaatggtggtgggcgcgccttaatgataaggagggatatgttccacgtaacttgctgggactgtacccaagaattaaaccaagacaaaggagcttggcctgaaacttccacacagaattttagtcaatgaagaattaatctctgttaagaagaagtaatacgattatttttggcaaaaatttcacaagacttattttaatgacaatgtagcttgaaagcgatgaagaatgtctctagaagagaatgaaggattgaagaattcaccattagaggacatttagcgtgatgaaataaagcatctacgtcagcaggccatactgtgttggggcaaaggtgtcccgtgtagcactcagataagtatacagcgacaatcctgttttctacaagaatcctgtctagtaaataggatcatttattgggcagttgggaaatcagctctctgtcctgttgagtgttttcagcagctgctcctaaaccagtcctcctgccagaaaggaccagtgccgtcacatcgctgtctctgattgtccccggcaccagcaggccttggggctcactgaaggctcgaaggcactgcacaccttgtatattgtcagtgaagaacgttagttggttgtcagtgaacaataactttattatatgagtttttgtagcatcttaagaattatacatatgtttgaaatattgaaactaagctacagtaccagtaattagatgtagaatcttgtttgtaggctgaattttaatctgtatttattgtcttttgtatctcagaaattagaaacttgctacagacttacccgtaatatttgtcaagatcatagctgactttaaaaacagttgtaataaactttttgatgct

SEQ ID NO: 2 - protein, 1005 aa, Homo sapiens
MDLTLAELQEMASRQQQQIEAQQQLLATKEQRLKFLKQQDQRQQQQVAEQEKLKRLKEIAENQEAKLKKVRALKGHVEQKRLSNGKLVEEIEQMNNLFQQKQRELVLAVSKVEELTRQLEMLKNGRIDSHHDNQSAVAELDRLYKELQLRNKLNQEQNAKLQQQRECLNKRNSEVAVMDKRVNELRDRLWKKKAALQQKENLPVSSDGNLPQQAASAPSRVAAVGPYIQSSTMPRMPSRPELLVKPALPDGSLVIQASEGPMKIQTLPNMRSGAASQTKGSKIHPVGPDWSPSNADLFPSQGSASVPQSTGNALDQVDDGEVPLREKEKKVRPFSMFDAVDQSNAPPSFGTLRKNQSSEDILRDAQVANKNVAKVPPPVPTKPKQINLPYFGQTNQPPSDIKPDGSSQQLSTVVPSMGTKPKPAGQQPRVLLSPSIPSVGQDQTLSPGSKQESPPAAAVRPFTPQPSKDTLLPPFRKPQTVAASSIYSMYTQQQAPGKNFQQAVQSALTKTHTRGPHFSSVYGKPVIAAAQNQQQHPENIYSNSQGKPGSPEPETEPVSSVQENHENERIPRPLSPTKLLPFLSNPYRNQSDADLEALRKKLSNAPRPLKKRSSITEPEGPNGPNIQKLLYQRTTIAAMETISVPSYPSKSASVTASSESPVEIQNPYLHVEPEKEVVSLVPESLSPEDVGNASTENSDMPAPSPGLDYEPEGVPDNSPNLQNNPEEPNPEAPHVLDVYLEEYPPYPPPPYPSGEPEGPGEDSVSMRPPEITGQVSLPPGKRTNLRKTGSERIAHGMRVKFNPLALLLDSSLEGEFDLVQRIIYEVDDPSLPNDEGITALHNAVCAGHTEIVKFLVQFGVNVNAADSDGWTPLHCAASCNNVQVCKFLVESGAAVFAMTYSDMQTAADKCEEMEEGYTQCSQFLYGVQEKMGIMNKGVIYALWDYEPQNDDELPMKEGDCMTIIHREDEDEIEWWWARLNDKEGYVPRNLLGLYPRIKPRQRSLA

SEQ ID NO: 3 - protein, 9 aa, Artificial Sequence
  P53 BP2
FLLWAEFTV

SEQ ID NO: 4 - DNA, 27 bp, Artificial Sequence
  P53 BP2
ttyytnytntgggcngarttyacngtn

SEQ ID NO: 5 - protein, 9 aa, Artificial Sequence
  P53 BP2
FLIRMEVMV

SEQ ID NO: 6 - DNA, 27 bp, Artificial Sequence
  P53 BP2
ttyytnathmgnatggargtnatggtn

SEQ ID NO: 7 - protein, 9 aa, Artificial Sequence
  P52 BP2
FLVETEKEV

SEQ ID NO: 8 - DNA, 27 bp, Artificial Sequence
  P53 BP2
ttyytngtngaracngaraargargtn

SEQ ID NO: 9 - protein, 9 aa, Artificial Sequence
  P53 BP2
FLILGEFEV

SEQ ID NO: 10 - DNA, 27 bp, Artificial Sequence
  P53 BP2
ttyytnathytnggngarttygargtn

SEQ ID NO: 11 - protein, 9 aa, Artificial Sequence
  P53 BP2
FLNEWIVMV

SEQ ID NO: 12 - DNA, 27 bp, Artificial Sequence
  P52 BP2
ttyytnaaygartggathgtnatggtn

SEQ ID NO: 13 - protein, 9 aa, Artificial Sequence
  P53 BP2
FLLDQPQFV

SEQ ID NO: 14 - DNA, 27 bp, Artificial Sequence
  P53 BP2
ttyytnytngaycarccncarttygtn

SEQ ID NO: 15 - protein, 9 aa, Artificial Sequence
  P53 BP2
FLVWDETQV

SEQ ID NO: 16 - DNA, 27 bp, Artificial Sequence
  P53 BP2
ttyytngtntgggaygaracncargtn

SEQ ID NO: 17 - protein, 9 aa, Artificial Sequence
  P53 BP2
FLVDFEHMV

SEQ ID NO: 18 - DNA, 27 bp, Artificial Sequence
  P53 BP2
ttyytngtngayttygarcayatggtn

SEQ ID NO: 19 - protein, 9 aa, Artificial Sequence
  P53 BP2
FLIYGEAEV

SEQ ID NO: 20 - DNA, 27 bp, Artificial Sequence
  P53 BP2
ttyytnathtayggngargcngargtn

SEQ ID NO: 21 - protein, 9 aa, Homo sapiens
LHVEPEKEV

(19) United States
(12) Patent Application Publication
(10) Pub. No.: US 2006/0099202 A1
(43) Pub. Date: May 11, 2006

(54) Immunoglobulin construct containing tumor- specific p53bp2 sequenes for eliciting an anti-tumor response

(75) Inventors: Charles A Nicolette, Framington, MA (US); Daniel A Soltis, Belle Mead, NJ (US)

Correspondence Address: DARBY & DARBY P.C., P. O. BOX 5257, NEW YORK, NY, 10150-5257, US

(73) Assignee: Euro-Celtique S.A., Luxembourg (LU)

(21) Appl. No.: 10/473,691

(22) PCT Filed: Apr. 1, 2002
(86) PCT No.: PCT/US02/10224

§ 371 Date: Apr. 23, 2004

Related U.S. Application Data
(60) Provisional application No. 60/280,733, filed on Mar. 30, 2001.

(51) Int. Cl. A61K 39/395; C07K 16/44

(52) U.S. Cl. 424/133.1; 530/387.3

(57) ABSTRACT

The present invention provides immunoglobulins specific for p53BP2 ligand polypeptides. In a preferred embodiment, the present invention provides a variant of an immunoglobulin variable domain, wherein the immunoglobulin variable domain contains (A) at least one CDR region and (B) framework regions flanking the CDR, and the variant includes: (a) the CDR region having added or substituted therein at least one binding sequence, and (b) the flaking framework regions, wherein the binding sequence is heterologous to the CDR and the binding sequence is derived from a human ligand having immunogenic properties relevant to human lung cancer. In a preferred embodiment, the binding sequence is an antigenic sequence. In a further preferred embodiment, the variant contains a variable domain lacking an intrachain disulfide bond.

### CROSS-REFERENCE TO PREVIOUS APPLICATIONS

This application claims priority from U.S. provisional patent application Ser. No. 60/280,733 filed Mar. 30, 2001.

### FIELD OF THE INVENTION

The present invention relates to products and methods to treat cancer to elicit an immune response targeted to tumor cells that bear a novel tumor antigen. In particular, the invention relates to immunoglobulin-derived variant constructs that carry antigenic sequences and use of such variant constructs for immunotherapy of cancer.

### BACKGROUND OF THE INVENTION

Cancer remains the second leading cause of death in the United States. There were an estimated 563,100 cancer deaths in 1999. Each year, about 1,222,000 new cancer cases are diagnosed

Non-surgical therapy for breast, lung, colon, and ovarian cancers, as well as many other solid tumors, is presently poor. While initial therapies for breast and ovarian cancer with taxanes result in some response by most patients, nearly all patients with ovarian cancer and some patients with breast cancer relapse.

There is substantial evidence indicating that the immune system plays a critical role in the prevention of cancer and the control of tumor growth. This includes the occasional observation of spontaneous tumor regression, the correlation of spontaneous regressions with the presence of tumor-infiltrating lymphocytes (TILs) and the identification of TILs that are specific for tumor antigens. However, as evidenced by the incidence rates of cancer, the immune response is often not sufficient to successfully combat the tumor. During the past 40 years, an increased understanding of the immune mechanism has led to the development of approaches to enhance the immune response against the tumor. These have included the expression in tumor cells of genes encoding immunostimulatory molecules and several approaches to enhance tumor antigen presentation. Unfortunately, only limited indications of beneficial effects have been seen and attempts to utilize these new approaches to immunotherapy have been disappointing.

### Immunoglobulins and Anti-Idiotype Immune Response

The basic unit of antibody immunoglobulin structure is a complex of four polypeptides—two identical low molecular weight or “light” chains and two identical high molecular weight or “heavy” chains—linked together by both non-covalent associations and by disulfide bonds. Each light and heavy chain of an antibody has a variable region at its amino terminus and a constant domain at its carboxyl terminus. The variable regions are distinct for each antibody and contain the antigen binding site. Each variable domain is comprised of four relatively conserved framework regions and three regions of sequence hypervariability termed complementarity determining regions or “CDRs”. For the most part, it is the CDRs that form the antigen binding site and confer antigen specificity. The constant domains are more highly conserved than the variable regions, with slight variations due to haplotypic differences.

Based on their amino acid sequences, light chains are classified as either kappa or lambda. The constant region of heavy chains is composed of multiple domains (CH1, CH2, CH3 . . . CHx), the number depending upon the particular antibody class. The CH1 region is separated from the CH2 region by a hinge region that allows flexibility in the antibody. The variable region of each light chain aligns with the variable region of each heavy chain, and the constant region of each light chain aligns with the first constant region of each heavy chain. The CH2-CHx domains of the constant region of a heavy chain form an “Fc region” that is responsible for the effector functions of the immunoglobulin molecule, such as complement binding and binding to the Fc receptors expressed by lymphocytes, granulocytes, monocyte lineage cells, killer cells, mast cells, and other immune effector cells.

In modern medicine, immunotherapy or vaccination has virtually eradicated diseases such as polio, tetanus, tuberculosis, chicken pox, measles, hepatitis, etc. The approach using vaccinations has exploited the ability of the immune system to prevent infectious diseases.

There are two types of immunotherapy, active immunotherapy and passive immunotherapy. In active immunotherapy, an antigen is administered in a vaccine to a patient so as to elicit a long-lasting protective immune response against the antigen. Passive immunotherapy involves the administration of protective antibodies to a patient to elicit an acute immune response that lasts only as long as the antibody is present. Antibody therapy is conventionally characterized as passive since the patient is not the source of the antibodies. However, the term passive may be misleading because a patient may produce antiidiotype secondary antibodies, which in turn provoke an immune response that is cross-reactive with the original antigen. Immunotherapy where the patient generates antiidiotypic antibodies is often more therapeutically effective than passive immunotherapy because the patient's own immune system generates an active immune response against cells bearing the particular antigen well after the initial infusion of protective antibody has cleared from the system.

In an anti-idiotypic response, antibodies produced initially during an immune response or introduced into an organism will carry unique new epitopes to which the organism is not tolerant, and therefore will elicit production of secondary antibodies (termed “Ab2”), some of which are directed against the idiotype (i.e., the antigen binding site) of the primary antibody (termed “Ab1”), i.e., the antibody that was initially produced or introduced exogenously. These secondary antibodies or Ab2, likewise will have an idiotype that will induce production of tertiary antibodies (termed “Ab3”), some of which will recognize the antigen binding site of Ab2, and so forth. This is known as the “network” theory. Some of the secondary antibodies will have a binding site that is an analog of the original antigen, and thus will reproduce the “internal image” of the original antigen. Tertiary or Ab3 antibodies that recognize this antigen binding site of the Ab2 antibody will also recognize the original antigen.

Therefore, anti-idiotypic antibodies have binding sites that are similar in conformation and charge to the antigen, and can elicit the same or a greater response than that of the target antigen itself. Administration of an exogenous antibody that can elicit a strong anti-idiotypic response can thus serve as an effective vaccine, by eliciting a self-propagating anti-idiotype immune response.

Clinically useful anti-idiotypic responses are rare when intact antibodies are used as the immunogen. The ability to deliver antibodies that reproducibly cause the generation of such an anti-idiotypic response is difficult (Foon, et al., J. Clin. Invest. 1995, 9:334-342; Madiyalakan, et al., Hybridoma 1995, 14: 199-203). One of the reasons for the failure generally to generate an anti-idiotypic response is that Ab1, while exogenous, is still very similar to “self”, as all antibodies have very similar structure and anti-idiotypic responses to self molecules tend to be very limited. A strong anti-idiotypic cascade has been observed when Ab1 has been structurally damaged (Madiyalakan et al., Hybridoma, 1995, 14:199-203) rendering the antibody more foreign. U.S. Pat. No. 4,918,164 discloses direct administration to the subject of exogenously produced anti-idiotype antibodies that are raised against the idiotype of an anti-tumor antibody. After administration, the subject produces anti-antibodies that not only recognize these anti-idiotype antibodies, but also recognize the original tumor epitope, thereby directing complement activation and other immune system responses to a foreign entity to attack the tumor cell that expresses the tumor epitope.

PCT Publication WO 99/25378 relates to synthebody molecules, particularly antibodies, that bind one member of a binding pair and have at least one complementarity determining region (CDR) that contains the amino acid sequence of a binding site for that member of the binding pair. The binding site is derived from the other member of the binding pair. It also relates to methods for treating, diagnosing, or screening for diseases and disorders associated with the expression of the member of the binding pair using the modified antibodies.

PCT Publication WO 99/25379 relates to vaccine compositions of antibodies in which one or more variable region cysteine residues that form intrachain disulfide bonds have been replaced with amino acid residues that do not contain a sulfhydryl group and, therefore, do not form disulfide bonds. It also relates to use of the vaccine compositions to treat or prevent certain diseases and disorders.

### Immunotherapy of Cancer

Immunotherapeutic strategies have been developed that attempt to “modulate” various aspects of the immune response associated with cancer. Many of these approaches depend in part upon the use of identified and characterized tumor-specific antigens.

In one strategic area, manipulation of antibody molecules and humoral immune responses directed against normal or mutated cellular antigens expressed in cancers or virally infected cells provide therapeutic and diagnostic agents. Vaccines can product antibodies directed against tumor specific antigens for immunotherapy to produce antibody-dependent cellular cytotoxicity, complement-dependent cytolysis, and apoptosis (Sinkovics and Horvath, Int. J. Oncol. 2000, 16:81-96; Weiner, Semin. Oncol. 1999, 26:43-51). Antibody immuno-conjugates derived from tumor antigen specific monoclonal antibodies are useful as delivery agents for cytotoxic agents and radionuclides or as imaging agents for diagnostic applications (Roselli et al., Anticancer Res. 1996, 16(4B):2187-2192; Trial and Bianchi Curr Opin Immunol. 1999, 11:584-588). Anti-tumor anti-bodies are those that induce anti-idiotype antibodies that mimic characteristics of tumor antigens and which are capable of further inducing anti-tumor humoral and cellular immune responses against tumors (Fagerberg et al. Proc Natl Acad Sci USA 1995, 92:4773-4777).

In cancer, tumor-specific T cells that are capable of binding lysing tumor cells displaying the corresponding tumor-associated antigen on their cell surfaces can be derived from patients. Tumor-specific T cells are localized at several sites within cancer patients, including in the blood (where they can be found in the peripheral and mononuclear cell fractions), in primary and secondary lymphoid tissue, i.e., the spleen, in ascites fluid in ovarian cancer patients (tumor-associated lymphocytes or “TALS”), or within the tumor itself (tumor infiltrating lymphocytes or “TILS”). Of these T cells populations, TILs have been the most useful in the identification of tumor antigens and epitopes thereof.

The specificity of tumor-specific cells is based on the ability of the T cell receptor (TCR) to recognize and bind to a short amino acid sequence which is presented on the surface of the tumor cells by MHC class I and, in some cell types, class II molecules. In brief, these amino acid binding sequences (also termed “ligands” or “epitopes”) are derived from the proteolytic degradation of intracellular proteins encoded by genes that are either uniquely or aberrantly expressed in tumor or cancer cells. The peptide ligands and intracellular proteins containing these epitopes are designated “tumor antigens”.

Conventional methods for culturing and subcloning of tumor-specific T cells are known in the art. Once a potent anti-tumor T cell population is recovered, it can be used to identify tumor antigens via conventional, but often tedious, expression cloning methodology (Kawakami Y. et al., Proc. Natl. Acad. Sci. USA 1994, 91:3515-3519). The results of numerous attempts to use expression cloning for generating tumor-specific T cells in vitro, however, suggest that this methodology is unreliable.

The availability of tumor-specific T cells has facilitated the identification of some tumor antigens, which have been employed in cancer vaccine compositions with varying degrees of success. Attempts to provoke anti-tumor responses in vivo by vaccination with protein or peptide fragments, however, are often unsuccessful, presumably because the protein or peptide fragments fail to access the cytosol of a cell and, therefore are not properly processed and presented to effector cells.

While it is often possible to identify the T cell epitope of a known antigen polypeptide, the same does not hold true for the identification of novel native antigens and epitopes. The application of conventional methodology has been sufficient to address and/or overcome the considerable complexities and variables associated with challenge to skilled artisans in the filed of the invention.

Thus, a need exists for additional novel cancer antigens and identification of the corresponding epitopes, and for effective delivery of these cancer antigens to provoke an anti-tumor immune response. Identified, characterized and isolated novel antigens and epitopes have substantial utility in therapeutic, prophylactic and diagnostic applications, in developing alternative immunogenic versions thereof, as well as in compositions and methods to induce beneficial immune responses.

In sum, there is a need in the art to identify effective methods to target tumor cells for immune clearance. In particular, there is a need to generate an effective anti-tumor immune response. The present invention addresses these and other needs in the art with the discovery of an effective therapy based on immunoglobulin variant constructs carrying immunogenic tumor antigen sequences.

### SUMMARY OF THE INVENTION

The present invention provides immunoglobulins specific for p53BP2 ligand polypeptides. In a preferred embodiment, the present invention provides a variant of an immunoglobulin variable domain, wherein the immunoglobulin variable domain contains (A) at least one CDR region and (B) framework regions flanking the CDR, and the variant includes: (a) the CDR region having added or substituted therein at least one binding sequence, and (b) the flanking framework regions, wherein the binding sequence is heterologous to the CDR and the binding sequence is derived from a human ligand having immunogenic properties relevant to human lung cancer. In a preferred embodiment, the binding sequence is an antigenic sequence. In a further preferred embodiment, the variant contains a variable domain lacking an intrachain disulfide bond.

The variant described above may also include one or more of the following: (i) one or more amino acid residues in one or more of said flanking framework regions has been substituted or deleted, (ii) one or more amino acid residues has been added in one or more of said flanking framework regions, or (iii) a combination of (i) and (ii).

Alternatively, the variant described above may also include one or more of the following additional features: (i) one or more amino acid residues in one or more framework regions other than said framework regions flanking said CDR has been substituted or deleted, (ii) one or more amino acid residues has been added in one or more framework regions other than said framework regions flanking said CDR, or (iii) a combination of (i) and (ii).

The invention also provides a variant as defined above wherein (i) one or more amino acid residues in one or more of said flanking framework regions has been substituted or deleted, (ii) one or more amino acid residues has been added in one or more of said flanking framework regions, or (iii) a combination of (i) and (ii); and wherein (iv) one or more amino acid residues in one or more framework regions other than said framework regions flanking said CDR has been substituted or deleted, (v) one or more amino acid residues has been added in one or more framework regions other than said framework regions flanking said CDR, or (vi) a combination of (iv) and (v).

In a further embodiment, the invention provides a variant of an immunoglobulin variable domain, wherein the immunoglobulin variable domain includes (A) at least one CDR region and (B) framework regions flanking the CDR, the variant containing:

(a) the CDR region having added or substituted therein at least one amino acid sequence which is heterologous to the CDR, and

(b) the flanking framework regions,

wherein the heterologous sequence is an antigenic sequence from a human ligand having immunogenic properties relevant to human lung cancer. In a preferred embodiment, the variable domain lacks an intrachain disulfide bond. The variant may also include one or more of the following: (i) one or more amino acid residues in one or more of said flanking framework regions has been substituted or deleted, (ii) one or more amino acid residues has been added in on or more of said flanking framework regions, or (iii) a combination of (i) and (ii). Alternatively, the variant may include: (i) one or more amino acid residues in one or more framework regions other than said framework regions flanking said CDR has been substituted or deleted, (ii) one or more amino acid residues has been added in one or more framework regions other than said framework regions flanking said CDR, or (iii) a combiantion of (i) and (ii). The variant may also contain (i) one or more amino acid residues in one or more of said flanking framework regions has been substituted or deleted, (ii) one or more amino acid residues has been added in one or more of said flanking framework regions, (iii) a combination of (i) and (ii); and wherein (iv) one or more amino acid residues in one or more framework regions other than said framework regions flanking said CDR has been substituted or deleted, (v) one or more amino acid residues has been added in one or more framework regions other than said framework regions flanking said CDR, or (vi) a combination of (iv) and (v).

The variant described above may contain more than one CDR.

The variant of the invention may also contain a heterologous sequence that is a CDR of a heavy chain variable region or a light chain variable region.

The variant of the invention may contain antigenic sequence having the amino acid sequences selected from the group consisting of FLLWAEFTV (SEQ ID NO:3), FIRMEVMV (SEQ ID NO:5), FLVETEKEV (SEQ ID NO:7), FLILGEFEV (SEQ ID NO: 9), FLNEWIVMV (SEQ ID NO:11), FLLDQPQFV (SEQ ID NO:13), FLVWDETQV (SEQ ID NO:15), FLVDFEHMV (SEQ ID NO:17), FLIYGEAEV (SEQ ID NO:19), and LHVEPEKEV (amino acids 669 to 677 of SEQ ID NO:2, also SEQ ID NO: 21).

The invention also provides a variant as defined above which is an antibody.

In addition, the invention provides a molecule containing the variant(s) described above. The molecule may be derived from a human antibody, a chimeric or a humanized antibody.

The invention also provides an immunuglobulin containing a heavy chain and a light chain, wherein said heavy chain comprises a variant as described above and three constant domains from an immunoglobulin heavy chain, and said light chain comprises a second variable domain associated with said variant and a constant domain from an immunoglobulin light chain. The invention further provides an immunoglobulin containing a heavy chain and a light chain, wherein said light chain comprises a variant as defined above and a constant domain from an immunoglobulin light chain, and said heavy chain comprises a second variable domain associated with said variant and three constant domains from an immunoglobulin heavy chain.

Moreover, the invention provides a variant of an immunoglobulin variable domain, said immunoglobulin variable domain comprising at least one CDR region, said variant comprising said CDR region having added or substituted therein at least one antigenic sequence from a human ligand having immunogenic properties relevant to human lung cancer, said at least one sequence being selected from the group consisting of (a) a binding sequence heterologous to said CDR; (b) a CTL-epitope sequence; (c) a T-helper cell sequence; (d) a B-helper cell sequence; and (e) combinations thereof, wherein said at least one sequence is heterologous to said CDR and the variable domain lacks an intrachain disulfide bond. In a preferred embodiment, the variable region comprises (a) a CDR1 region having said CTL epitope sequence substituted or added therein; (b) a CDR2 region having said T-helper cell substituted or added therein; and (c) a CDR3 region having said binding sequence of B-helper cell sequence substituted or added therein. This variant may be an antibody, and the invention also contemplates incorporating such a variant in a molecule, which may further comprise one or more constant domains from an immunoglobulin, a second variable domain linked to said variant, and/or a second variable domain linked to said variant and one or more constant domains from an immunoglobulin. The molecule may be an antibody, and it may also be derived from a human antibody or from a chimeric or humanized antibody. Also provided is an immunoglobulin comprising a heavy chain and a light chain, wherein said heavy chain comprises a variant described above and three constant domains from an immunoglobulin heavy chain, and said light chain comprises a second variable domain associated with said variant and a constant domain from an immunoglobulin light chain; alternatively, the invention provides an immunoglobulin comprising a heavy chain and a light chain, wherein said light chain comprises a variant as described above and a constant domain from an immunoglobulin light chain, and said heavy chain comprises a second variable domain associated with said variant and three constant domains from an immunoglobulin heavy chain.

In a further embodiment, the invention provides an isolated nucleic acid encoding encoding a variant or immunoglobulin as described above. Also provided is a cell and/or recombinant non-human host containing such a nucleic acid.

The invention also provides a vaccine composition containing a therapeutically or prophylactically effective amount of a variant, molecule, immunoglobulin, nucleic acid, or cell as defined above and an adjuvant.

The constructs of the invention may be used in a method of treating or preventing a lung cancer tumor in a subject in need of such treatment or prevention, said method comprising administering to said subject a disease treating or preventing effective amount of a variant, molecule, immunoglobulin, nucleic acid, cell, or vaccine, as described above.

Finally, the invention provides a method of eliciting an anti-idiotypic response to a tumor antigen in a subject in need of treatment or prevention of a disease condition associated with said tumor antigen, said method comprising administering to said subject a disease treating or preventing effective amount of a variant, molecule, immunogloboulin, nucleic acid, cell, or vaccine, as described above.

### Description of the Sequence Listings

SEQ ID NO:1. The complete nucleotide sequence of a cDNA encoding the human cancer antigen p53BP2 (GenBank Accession No. 005417.1). The coding region extends from nucleotide 1 through nucleotide 4534.

SEQ ID NO:2. The amino acid sequence (1005 amino acids) of the native human cancer antigen p53BP2. The compounds of the invention are variations of a peptide comprising amino acids 669-677 (also SEQ ID NO 21).

SEQ ID NO:3. The amino acid sequence of compound 1.

SEQ ID NO:4. The polynucleotide sequence encoding compound 1.

SEQ ID NO:5. The amino acid sequence of compound 2.

SEQ ID NO:6. The polynucleotide sequence encoding compound 2.

SEQ ID NO:7. The amino acid sequence of compound 3.

SEQ ID NO:8. The polynucleotide sequence encoding compound 3.

SEQ ID NO:9. The amino acid sequence of compound 4.

SEQ ID NO:10. The polynucleotide sequence encoding compound 4.

SEQ ID NO:11. The amino acid sequence of compound 5.

SEQ ID NO:12. The polynucleocide sequence of compound 5.

SEQ ID NO:13. The amino acid sequence of compound 6.

SEQ ID NO:14. The polynucleotide sequence encoding compound 6.

SEQ ID NO:15. The amino acid sequence of compound 7.

SEQ ID NO:16. The polynucleotide sequence encoding compound 7.

SEQ ID NO:17. The amino acid sequence of compound 8.

SEQ ID NO:18. The polynucleotide sequence encoding compound 8.

SEQ ID NO:19. The amino acid sequence of compound 9.

SEQ ID NO:20. The polynucleotide sequence encoding compound 9.

SEQ ID NO: 21: Amino acids 669 to 677 of the native human cancer antigen p53BP2.

### DESCRIPTION OF THE DRAWINGS

FIGS. 1A and 1B. Consensus amino acid sequences of (A) the heavy chain variable region and (B) the light chain variable region.

FIG. 2. Diagram of PCR knitting strategy.

### DETAILED DESCRIPTION

The present invention provides an approach to elicit an anti-tumor immune response. The invention involves introducing antigenic sequences from a ligand having immunogenic properties relevant to human lung cancer into a synthetic construct, e.g., designed to elicit an anti-idiotypic immune response. In particular, Ab2 antibodies in an anti-idiotype pathway target ligand-bearing tumor cells for destruction.

Thus, in one aspect, the invention provides a construct that contains an antigenic sequence or sequences from a ligand having immunogenic properties relevant to human lung cancer in the CDR positions, preferably with a disrupted intrachain disulfide bond in the variable domain (which contains the CDRs), which CDRs are each flanked by framework regions of a variable region. In addition, the invention provides constructs comprising (A) at least one CDR region having added or substituted therein at least one amino acid sequence which is heterologous to the CDR and the flanking framework regions, wherein the heterologous sequence is capable of binding to a target sequence or molecule, and wherein the heterologous sequence is an antigenic sequence from a ligand having immunogenic properties relevant to human lung cancer. In addition, the CDR may also include at least one amino acid sequence selected from (a) a cytotoxic lymphocyte (CTL) epitope sequence, (b) a T-helper cell sequence, (c) a B-cell epitope sequence, (d) a targeting sequence, or combinations thereof, and the variant preferably includes a disrupted intrachain disulfide bond. Each of the CTL, T-helper cell, B-cell epitope, and targeting sequences are antigenic sequences from a ligand having immunogenic properties relevant to human lung cancer. Preferably, the construct consists of (a) a CTL epitope sequence substituted or added to CDR1, (b) a T-helper cell sequence substituted or added to CDR2, and (c) at least one amino acid sequence which is heterologous to the CDR and the flanking framework regions, wherein the heterologous sequence is capable of binding to a target sequence or molecule, and wherein the heterologous sequence is an antigenic sequence from a ligand having immunogenic properties relevant to human lung cancer. Alternatively, the construct comprises (a) a CTL epitope sequence substituted or added to CDR1, (b) a T-helper cell sequence substituted or added to CDR2, and (c) a B-cell epitope sequence substituted or added to CDR3.

The invention further provides pharmaceutical and vaccine compositions that comprise an amount of such a construct effective to elicit an anti-tumor Ab2 anti-idiotype response ill vivo, a pharmaceutically acceptable carrier or excipient, and optimally an adjuvant.

Recombinant nucleic acids, particularly DNA molecules, provide for efficient expression of the foregoing constructs. In one specific aspect of this embodiment, the invention provides a nucleic acid encoding the synthebody. Also encompassed are expression vectors in which the nucleic acid is operably associated with an expression control sequence. The invention extends to host cells transfected or transformed with the expression vector. The construct can be produced by isolating it from the host cells grown under conditions that permit expression of the synthebody.

The invention also furnishes a vaccine composition comprising the vector that expresses the construct in an amount effective to produce sufficient construct to elicit an anti-tumor Ab2 response.

The present invention is based on discovery of the p53BP2 ligand, and construction of synthetic modified antibodies containing antigenic ligand sequences. Using oligodeoxyribonucleotides, heavy and light chain variable region genes were constructed to contain sequences from ligand p53BP2 in the CDR regions. The engineered variable region was then inserted into a mammalian expression vector containing the appropriate constant regions. A vector containing both light and heavy chains was transfected into a mammalian cell and the synthetic antibody was expressed.

The primary activity of the synthetic construct is to elicit an anti-tumor anti-idiotype immune response in order to decrease the rate of tumor growth, cause tumor regression, increase the time to relapse, and decrease mortality. The slower clearance seen with antibodies and other immunoglobulin superfamily proteins combined with an enhanced ability to elicit an anti-idiotypic antibody response gives the constructs an advantage for cancer therapy in vivo.

The term “construct” refers to the variant of a variable domain of an immunoglobulin superfamily protein, including molecules comprising such variants, described herein. The immunoglobulin superfamily is well known, and includes antibody/B-cell receptor proteins, T lymphocyte receptor proteins, and other proteins mentioned infra (see, Paul, Fundamental Immunology, 3^{rd} Ed.). The modification refers to insertion into or substitution of a portion of the immunoglobulin superfamily protein sequence with a heterologous amino acid sequence or heterologous binding sequence. The site of substitution in the immunoglobulin superfamily protein corresponds to a binding-accessible portion of the region of the immunoglobulin superfamily protein, e.g., a region that corresponds to an antibody variable region, and more particularly a portion corresponding to a CDR of an antibody variable region.

A “synthebody” (for synthetic antibody) is a specific example of a construct of the invention that includes an antibody variable region. It may also include regions corresponding to an antibody constant region or regions, or be associated with one or more other immunoglobulin family polypeptides, such as an antibody Fv heterodimer, an antibody tetramer, a T lymphocyte receptor heterodimer, etc. Embodiments described below are illustrative of the variants and molecules of the present invention in that the variants are included in synthebodies and synthebodies are a type of molecule that includes the variants. The term “synthebody” thus refers to an illustrative example of a type of construct of the invention.

The term “epitope string” refers to a molecule consisting of at least one epitope sequence and at least one targeting sequence and/or targeting mechanism. The epitope is preferably derived from a tumor associated antigen. For example, the epitope sequence may include a CD8+, CD4+, and/or B cell epitopes. The epitope strings of the invention comprise at least one epitope sequence, or a combination of epitope sequences, which may be the same or different.

A “targeting mechanism” is a system that is capable of directing the epitope strings of the present invention to antigen presenting cells and facilitating proper internal processing of the constructs once inside the antigen presenting cells. Thus, in one embodiment of the present invention, the targeting mechanism includes a “targeting sequence” which is comprised of two components: (1) an antigen-presenting cell targeting sequence, which is specifically tailored to ensure that the constructs of the invention reach the antigen presenting cells and/or it preserves the viability of the construct until it has reached the antigen presenting cells; and (2) an internal processing sequence, which is designed to facilitate endosomal and/or lysosomal processing of the construct once it has been internalized by the antigen presenting cell. In addition, the “targeting mechanism” may also include the administration of the epitope strings of the invention in a targeting vehicle, e.g., a microsphere or liposome, which ensures that the epitope strings reach their intended target and remain viable until that time.

In a preferred embodiment, the antigen presenting cell targeting sequence comprises the p80 fragment of the gp96 alpha subunit. This “p80 sequence” preferably binds to CD91, a receptor for heat shock proteins. See Binder et al., “CD91: A Receptor for Heat Shock Protein”, Nature Immuno. 1: 151-55 (2000). The antigen presenting targeting sequence may be incorporated into the epitope string construct at the N- or C-terminus. In a preferred embodiment, the antigen-presenting targeting sequence is incorporated into the constructs of the invention at the C-terminus.

The internal processing sequence is designed to enable the epitope strings of the invention to be resistant to degradation by endogenous enzymes other than proteasomes. In addition, an internal processing sequence may catalyze ubiquitination of the eptiope strings in order that they may be recognized and processed by endosomes and/or lysosomes within antigen presenting cells. In this regard, specific residues that may be included in the internal processing sequence include, but are not limited to, arginine (Arg), leucine (leu), phenylalanne (Phe), aspartic acid (Asp), lysine (Lys), or proline (Pro). In a preferred embodiment, the internal processing sequence includes one or more Arg, Asp, and Pro residues.

The internal processing sequence also includes a “recognition sequence” that comprises one or more of the residues identified above that are recognized by endosomes or lysosomes. The recognition sequence should be followed by an “internal sequence” which contains at least one lysine residue, and preferably two lysine residues at positions 3 and 17 of the internal sequence. Immediately following or adjacent to the internal sequence is the sequence of the epitope. One or more targeting sequences may be incorporated into the epitope strings of the invention, the one or more targeting sequences being positioned in between each epitope sequence. With regard to the targeting sequence described above, reference is made to Bachmair et al., “In vivo Half-life of a Protein is a Function of its Amino-terminal Residue”, Science 234: 179-86 (1986); and Dantuma et al., “Short-lived Green Fluorescent Proteins for Quantifying Ubiquitin/Proteasome-dependent Proteolysis in living cells”, Nature Biotech. 18: 53843 (2000).

Further, endosomes and lysosomes possess proteolytic activity with substrate specificity similar to trypsin and chymotrypsin (see Hershko et al., “The Ubiquitin System”, Ann. Rev. Biochem. 67: 425-79 (1998)). Thus, the MHC Class I epitope sequences included in the epitope strings of the present invention are preferably bounded by amino acid residues that act as substrates for those enzymes, such as lysine or arginine for trypsin, and phenylalanine, tryptophan, or tyrosine for chymotrypsin.

Thus, a epitope strings of the present invention may be represented by the following formula:
[targeting sequence]-[epitope]-[targeting sequence]-[epitope] . . . [epitope][targeting sequence]
wherein the targeting sequence is:
N-[internal processing sequence]-[antigen presenting sequence]-C
such as
[(Leu, Asp, or Pro]-[Xaa-Lys-Xaa-Lys-Y_{T/C}],-[p80]
wherein Xaa is an amino acid, and Y_{T/C} is an amino acid that is susceptible to cleavage by trypsin or chymotrypsin.

The epitope sequences used in the epitope string constructs of the present invention may be any practical number and may be the same or different, and they may be repeats of a single epitope or several epitopes. The epitopes within an epitope string construct of the invention may be B-cell epitopes, CTL epitopes, T-helper epitopes, or combinations thereof.

Alternatively or additionally, the targeting mechanism of the invention may include incorporating the eptiope strings of the present invention into microspheres that are internalized, e.g., phagocytosed, by the antigen presenting cells, and then further processed by endosomes and/or lysosomes within the cells. In this regard, reference is made to U.S. Pat. No. 6,312,731 to Staas et al. (“Staas”). Staas relates to a composition for inducing or potentiating an immune response, comprising an antigen and/or a bioactive agent capable of inducing or potentiating an immune response, wherein the antibody or agent is encapsulated in a polymeric composition consisting of a polymer present in an amount sufficient to provide structural integrity to the composition, and a rapidly biodegradable component, a rapidly dissolving component, a rapidly swelling component, or a component that causes osmotic rupture of the encapsulated polymeric composition.

The term “heterologous” refers to a combination of elements not naturally occurring in a particular locus. For example, heterologous DNA refers to DNA not naturally located in the cell or in a particular chromosomal site of the cell. A heterologous expression regulatory element is such an element operatively associated with a different gene than the one it is operatively associated with in nature. In the context of the present invention, a construct coding sequence is heterologous to the vector DNA in which it is inserted for cloning or expression and it is heterologous to a host cell containing such a vector in which it is expressed, e.g., a CHO cell. Moreover, the constructs of the present invention contain a heterologous DNA, amino acid, or binding sequence.

The “heterologous amino acid sequence” (also “ligand sequence”) refers to the desired antigenic segment of a ligand polypeptide.

The term “CDR” refers to a part of the variable region of an immunoglobulin family protein that confers binding specificity, e.g., antibody specificity for antigen. In antibodies, CDRs are highly variable and accessible.

The term “framework region” refers to the part of the modified immunoglobulin molecule corresponding to an antibody framework region, as defined in the art. Sequences flanking the CDR are termed herein “framework regions of a variable region”.

The term “flanked” and “flanking” refers to the amino acids that are connected to or are connected by spacing amino acids to the protein sequence of the CDR. “Spacing amino acids” (or a “spacer group”) are amino acids that are not found in the native framework sequence or the CDR or the substituted sequence, nor do they independently confer any binding activity on the modified variable region. They may be included to preserve or ensure a proper variable region conformation and orientation of the CDR or substituted heterologous amino acid sequence.

The term “vaccine” refers to a composition (protein or vector; the latter may also be loosely termed a “DNA vaccine”, although RNA vectors can be used as well) that can be used to elicit protective immunity in a recipient. It should be noted that to be effective, a vaccine of the invention can elicit immunity in a portion of the population, as some individuals may fail to mount a robust or protective immune response, or, in some cases, any immune response. This inability may stem from the individual's genetic background or because of an immunodeficiency condition (either acquired or congenital) or immunosuppression (e.g., treatment with immunosuppressive drugs to prevent organ rejection or suppress an autoimmune condition). Efficacy can be established in animal models.

The term “immunotherapy” refers to a treatment regimen based on activation of a tumor-specific immune response. A vaccine can be one form of immunotherapy. Immunotherapy differs from vaccination in that the former usually refers to prophylaxis, while therapy can improve the course of a disease. The present invention permits both.

“Immune response” broadly refers to the antigen-specific responses of lymphocytes to foreign substances. Any substance that can elicit an immune response is said to be “immunogenic” and is referred to as an “immunogen”. All immunogens are antigens, however, not all antigens are immunogenic. An immune response of this invention can be humoral (via antibody activity) or cell-mediated (via T cell activation).

The terms “cancer,” “neoplasm,” and “tumor,” used interchangeably and in either the singular or plural form, refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but also any cell derived from a cancer cell ancestor. This includes metastasized cancer cells, and in vitro cultures and cell lines derived from cancer cells. When referring to a type of cancer that normally manifests as a solid tumor a “clinically detectable” tumor is one that is detectable on the basis of tumor mass; e.g., by such procedures as CAT scan, magnetic resonance imaging (MRI, X-ray, ultrasound or palpation. Biochemical or immunologic findings alone may be insufficient to meet this definition.

“Suppressing” tumor growth indicates a growth state that is curtailed compared to growth without contact with educated, antigen-specific immune effector cells described herein. Tumor cell growth can be assessed by any means known in the art, including, but not limited to, measuring tumor size, determining whether tumor cells are proliferating using a ³H-thymidine incorporation assay, or counting tumor cells. “Suppressing” tumor cell growth means any or all of the following states: slowing, delaying, and “suppressing” tumor growth indicates a growth state that is curtailed when stopping tumor growth, as well as tumor shrinkage.

The term “ligand” as used herein refers to any molecule that binds to a specific site on another molecule. In other words, the ligand confers the specificity of the protein in a reaction with an immune effector cell. It is the ligand site within the protein that combines directly with the complementary binding site on the immune effector cell. In a preferred embodiment, a ligand of the invention binds to an antigenic determinant or epitope on an immune effector cell, such as an antibody or a T cell receptor (TCR). A ligand may be antigen, peptide, protein or epitope having immunogenic properties relevant to human lung cancer. In one embodiment, the ligand of the invention is up to 100 amino acids in length. Furthermore, antigenic ligand sequences may bind to a receptor on an MHC class I molecule. In this embodiment, the antigenic ligand sequence of the invention is about 7 to about 11 amino acids in length. Antigenic ligand sequences may also bind to a receptor on an MHC class II molecule. In such an embodiment, the antigenic ligand sequence of the invention is about 10 to about 20 amino acids long.

As used herein, the term “immunogenic” means that the variant construct is capable of eliciting a humoral or cellular immune response, and preferably both. An immunogenic polypeptide is also antigenic. A molecule is “antigenic” when it is capable of specifically interacting with an antigen recognition molecule of the immune system, such as an immunoglobulin (antibody) or T cell antigen receptor. Antigenic ligand sequences that specifically interact with antibody, MCH class I, and MHC class II molecules are described herein. An antigenic portion of a polypeptide, also called herein the epitope, can be that portion that is immunodominant for antibody or T cell receptor recognition, or it can be a portion used to generate an antibody to the molecule by conjugating the antigenic portion to a carrier polypeptide for immunization. A molecule that is antigenic need not be itself immunogenic, i.e., capable of eliciting an immune response without a carrier.

A “native” or “natural protein” is a polypeptide which contains a ligand, which has been isolated from a natural biological source, and which can be recognized by the immune system. In this invention, native immunogenic ligands, which specifically bind to an antigen receptor, in particular a T cell antigen receptor (TCR), in a subject are of particular interest.

An “altered ligand” one having a primary sequence that is different from that of the corresponding native or “wild-type” ligand. Altered ligands, also known as a non-native-, modified-, and/or synthetic ligands, can be made by various means, and include, but are not limited to, antigen peptides that are differentially modified during or after translation, e.g. by phosphorylation, glycosylation, cross-linking, acylation, proteolytic cleavage, linkage to an antibody molecule, membrane molecule or other ligand (Ferguson et al. Ann. Rev. Biochem. 1988, 57:285-320). An altered ligand of the invention can be recognized and bound by an immune effector cell clone that is specific for the ligand of the corresponding native protein.

A “self-antigen” also referred to herein as a native or wild-type antigen is an antigenic peptide that induces little or no immune response in the subject due to self-tolerance to the antigen. An example of a self-antigen is the melanoma specific antigen gp100.

The term “tumor-associated antigen” or “TAA” refers to an antigenic peptide that is associated with or specific to a tumor and is presented to T cells by MHC molecules. Examples of known TAAs include gp100, MART and MAGE.

The terms “major histocompatability complex” or “MHC” refers to a complex of genes encoding cell-surface molecules that are required for antigen presentation to T cells and for rapid graft rejection. In humans, the MHC is also known as the “human leukocyte antigen” or “HLA” complex. The proteins encoded by the MHC are known as “MHC molecules” and are classified into class I and class II MHC molecules. Class I MHC includes membrane heterodimeric proteins made up of an α chain encoded in the MHC noncovalently linked with the β2-microglobulin. Class I MHC molecules are expressed by nearly all nucleated cells and have been shown to function in antigen presentation to CD8⁺ T cells. Class I molecules include HLA-A, B, and C in humans. Class II MHC molecules also include membrane heterodimeric proteins consisting of noncovalently associated α and β chains. Class II MHC molecules are known to function in CD4⁺ T cells and, in humans, include HLA-DP, -DQ, and DR. In a preferred embodiment, invention compositions and ligands can complex with MHC molecules of any HLA type. Those of skill in the art are familiar with the serotypes and genetypes of the HLA (see Rammensee, H. G., Bachmann, J., and Stevanovic, S. MHC Ligands and Peptid Motifs (1977) Chapman & Hall Publishers; Schreuder et al., The HLA dictionary, Tissue Antigens 1999, 54:409-437).

The term “antigen-presenting matrix”, as used herein, intends a molecule or molecules which can present antigen in such a way that the antigen can be bound by a T cell antigen receptor on the surface of a T cell. An antigen-presenting matrix can be on the surface of an antigen-presenting cell (APC), on a vesicle preparation of an APC, or can be in the form of a synthetic matrix on a solid support such as a bead or a plate. An example of a synthetic antigen-presenting matrix is purified MHC class I molecules complexed to β-microglobulin, multimers of such purified MHC class I molecules, purified MHC Class II molecules, or functional portions thereof, attached to a solid support.

The term “antigen presenting cells (APCs)” refers to a class of cells capable of processing one or more antigens and displaying fragments thereof in the for of a peptide-MHC complex on the cell surface together with costimulatory molecules required for lymphocyte activation. While many types of cells may be capable of presenting antigens on their cell surface for T cell recognition, only professional APCs have the capacity to present antigens in a efficient amount and further to activate T cells and imitate the cytolytic T cell response against the antigen. APCs can be intact whole cells such as macrophages, B-cells, and dendritic cells (DCs); or other molecules, naturally occurring or synthetic, such as purified MHC class I molecules complexed to β2-microglobulin.

The term “dendritic cells (DCs)” refers to a diverse population of morphologically similar cell types found in a variety of lymphoid and non-lymphoid tissues (Steinman, Ann. Rev. Immunol. 1991, 9:271-296). Dendritic cells constitute the most potent and preferred APCs in the organism. A subset, if not all, of dendritic cells are derived from bone marrow progenitor cells, circulate in small numbers in the peripheral blood and appear either as immature Langerhans' cells or terminally differentiated mature cells. While dendritic cells can be differentiated from monocytes, they posses distinct phenotypes. For example, a particular differentiating marker, CD14 antigen, is not found in dendritic cells but is possessed by monocytes. Also, mature dendritic cells are not phagocytic, whereas the monocytes are strongly phagocytosing cells. It has been shown that DCs provide all the signals necessary for T cell activation and proliferation.

The term “antigen presenting cell recruiting factors” of “APC” recruitment factors” include both intact, whole cells as well as other molecules that are capable of recruiting antigen presenting cells. Examples of suitable APC recruitment factors include molecules such as interleukin 5 (IL-4), granulocyte macrophage colony stimulating factor (GM-CSF), Sepragel and macrophage inflammatory protein 3 alpha (MIP3α). These are available from Immunex, Schering-Plough and R&D Systems (Minneapolis, Minn.). They also can be recombinantly produced using the methods disclosed in CURRENT PROTOCOLS IN MOLECULAR BIOLOGY (F. M. Ausubel et al., eds. (1987)). Peptides, proteins and compounds having the same biological activity as the above-note factors are included within the scope of this invention.

The term “immune effector cells” refers to cells capable of binding an antigen and which thereby mediate an immune response. These cells include, but are not limited to, T cells, B cells, monocytes, macrophages, NK cells and cytotoxic T lymphocytes (CTLs), for example CL lines, CTL clones, and CTLs from tumor, inflammatory, or other infiltrates. The activation of T cells by professional APCs leads to their proliferation and the differentiation of their progeny into armed effector T cells. Once an expanded clone of T cell achieves effector function, its armed effector T cell progeny can act on any target cell that displays antigen on its surface. Effector T cells can mediate a variety of functions. One set of important functions is the killing of infected cells by CD8⁺ CTLs and the activation of macrophages by T_{H}1 cells, which together make up cell-mediated immunity. A different function is the activation of B cells by both T_{H}2 and T_{H}1 cells to produce different types of antibody, thus driving the humoral immune response.

The term “immune effector molecule” as used herein, refers to molecules capable of antigen-specific binding, and includes antibodies, T cell antigen receptors, and MHC class I and class II molecules.

A “naïve” immune effector cell is an immune effector cell that has never been exposed to an antigen capable of activating that cell. Activation of naive immune effector cells requires both recognition of the peptide:MHC complex and the simultaneous delivery of a costimulatory signal by a professional APC in order to proliferate and differentiate into antigen-specific armed effector T cells.

As used herein, the term “educated, antigen-specific immune effector cell”, is an immune effector cell as defined above, which has previously encountered an antigen. In contrast with its naive counterpart, activation of an educated, antigen-specific immune effector cell does not require costimulatory signal. Recognition of the peptide:MHC complex is sufficient.

“Activated”, when used in reference to a T cell, implies that the cell is no longer in Gₒ phase, and begins to produce one or more of cytotoxins, cytokines, and other related membrane-associated proteins characteristic of the cell type (e.g., CB8⁺ or CD4⁺), is capable of recognizing and binding any target cell that displays the particular antigen on its surface, and releasing its effector molecules.

In the context of the present invention, the term “recognized” intends that a tumor cell, comprising one or more ligands, is recognized and bound by an immune effector cell wherein such binding initiates an effective immune response. Assays for determining whether a ligand is recognized by an immune effector cell are known in the art and are described herein.

The term “preferentially recognized” intends that the specificity of a ligand of the invention is restricted to immune effector cells that recognize and bind the native ligand.

The term “cross-reactive” is used to describe compounds of the invention which are functionally overlapping. More particularly, the immunogenic properties of a native ligand and/or immune effector cells activated thereby are shared to a certain extent by the altered ligand such that the altered ligand is “cross-reactive” with the native ligand and/or the immune effector cells activated thereby. For purposes of this invention, cross-reactivity is manifested at multiple levels: (i) at the ligand level, e.g., the altered ligands can bind the TCR of and activate native ligand CTLs; (ii) at the T cell level, i.e., altered ligands of the invention bind the TCR of and activate a population of T cells (distinct from the population of native ligand CTLs) which can effectively target and lyse cells displaying the native ligand; and (iii) at the antibody level, e.g., “anti”-altered ligand antibodies can detect, recognize and bind the native ligand and initiate effector mechanisms in an immune response which ultimately result in elimination of the native ligand from the host.

As used herein, the term “inducing an immune response in a subject” is a term well understood in the art and intends that an increase of at least about 2-fold, more preferably at least 5-fold, more preferably at least 10-fold, more preferably about 100-fold, even more preferably at least about 500-fold, even more preferably at least about 1000-fold or more in an immune response to an antigen (or epitope) can be detected or measured, after introducing the variant construct into the subject, relative to the immune response (if any) before introduction of the variant construct into the subject. An immune response to an antigen (or epitope), includes, but is not limited to, production of an antigen-specific (or epitope-specific) antibody, and production of an immune cell expressing on its surface a molecule which specifically binds to an antigen (or epitope). Methods of determining whether an immune response to a given antigen (or epitope) has been induced are well known in the art. For example, antigen-specific antibody can be detected using any of a variety of immunoassays known in the art, including, but not limited to, ELISA, wherein, for example, binding of an antibody in a sample to an immobilized antigen (or epitope) is detected with a detectably-labeled second antibody (e.g., enzyme-labeled mouse anti-human Ig antibody).

“Co-stimulatory molecules” are involved in the interaction between receptor-ligand pairs expressed on the surface of antigen presenting cells and T cells. Research accumulated over the past several years had demonstrated convincingly that resting T cells require at least two signals for induction of cytokine gene expression and proliferation (Schwartz, Science 1990, 248:1349-1356; Jenkins, [Immunol. Today 1992, 13:69-73). One signal, the one that confers specificity, ca be produced by interaction of the TCR/CD3 complex with an appropriate MHC/peptide complex. The second signal is not antigen specific and is termed the “co-stimulatory” signal. This signal was originally defined as an activity provided by bone-marrow derived accessory cells such as macrophages and dendritic cells, the so called “professional” APCs. Several molecules have been shown to enhance co-stimulatory activity. These are heat stable antigen (HAS) (Liu et al., J. Exp. Med. 1992, 175:437-445), chondroitin sulfate-modified MHC invariant chain (Li-CS) (Naujokas et al., Cell 1993, 73:257-267), intracellular adhesion molecule 1 (ICAM-1) (Van Seventer, J. Immunol. 1990, 144B:4579-4586), B7-1, and B7-2/B70 (Schwartz, Cell 1992, 71:1065-1068). These molecules each appear to assist co-stimulation by interacting with their cognate ligands on the T cells. Co-stimulatory molecules mediate co-stimulatory signal(s), which are necessary, under normal physiological conditions, to achieve full activation of naïve T cells. One exemplary receptor-ligand pair is the B7 co-stimulatory molecule on the surface of APCs and its counter-receptor CD28 or CTLA4 on T cells (Freeman et al., Science 1993, 262:909-911; Young et al., J. Clin. Invest. 1992, 90:229; and Nabavi et al., Nature 1992, 360:266-268). Other important co-stimulatory molecules” encompasses any single molecule or combination of molecules which, when acting together with a peptic/MHC complex bound by a TCR on the surface of a T cell, provides a co-stimulatory effect which achieves activation of the T cell that binds the peptide. The term thus encompasses B7, or other co-stimulatory molecule(s) on an antigen-presenting matrix such as an APC, fragments thereof (alone, complexed with another molecule(s), or as part of a fusion protein) which, together with peptide/MHC complex, binds to a cognate ligand and results in activation of the T cell when the TCR on the surface of the T cell specifically binds the peptide. Co-stimulatory molecules are commercially available from a variety of sources, including, for example, Beckman Coulter, Inc. (Fullerton, Calif.). It is intended, although not always explicitly stated, that molecules having similar biologically activity as wild-type or purified co-stimulatory molecules (e.g., recombinantly produced or muteins thereof) are intended to be used within the spirit and scope of the invention.

The term “protect” is used herein to mean prevent or treat, or both, as appropriate, a cancer, e.g., lung cancer, in a subject. Thus, prophylactic administration of the vaccine can protect the recipient subject at risk of developing such a cancer, e.g., as determined from family history.

The phrase “pharmaceutically acceptable”, whether used in connection with the pharmaceutical compositions of the invention or vaccine compositions of the invention, refers to molecular entities and compositions that are physiologically tolerable and do not typically produce untoward reactions when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like, including emulsions, such as an oil/water or water/oil emulsion. The pharmaceutical carriers can include various wetting agents, emulsifiers, stabilizers, preservatives, and the like. Water or aqueous solution saline solutions, particularly phosphate buffered saline solution, and aqueous dextrose and glycerol solutions are preferably employed as carriers, particularly for injectable solutions. Suitable pharmaceutical carriers are described in “Remington's Pharmaceutical Sciences” by E. W. Martin, 18^{th} Edition.

The term “adjuvant” refers to a compound or mixture that enhances the immune response to an antigen. An adjuvant can serve as a tissue depot that slowly releases the antigen and also as a lymphoid system activator that non-specifically enhances the immune response (Hood et al., Immunology, Second Ed., 1984, Benjamin/Cummings: Menlo Park, Calif., p. 384). Often, a primary challenge with an antigen alone, in the absence of an adjuvant, will fail to elicit a humoral or cellular immune response. Adjuvants include, but are not limited to, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, keyhole limpet hemocyanins, and potentially useful human adjuvants such as N-acetyl-muramyl-L-threonyl-D-isoglutamine (thr-MDP), N-acetyl-nor-muramyl-L-alanyl-D-isoglutamine, N-acetylmuramyl-L-alanyl-D-isoglutaminyl-L-alanine-2-(1′-2′-dipalmitoyl-sn-glycero-3-hydroxyphosphoryloxy)-ethylamine, cholera toxin, BCG (bacille Calmette-Guerin) and Corynebacterium parvum. Preferably, the adjuvant is pharmaceutically acceptable.

The term “immunomodulatory agent”, as used herein, is a molecule, a macromolecular complex, or a cell that modulates an immune response and encompasses compositions or ligands of the invention alone or in any of a variety of formulations described herein; a polypeptide comprising a ligand of the invention; a polynucleotide encoding a ligand or protein or the invention; a ligand of the invention bound to class I or a class II MHC molecule on an antigen-presenting matrix, including an APC and a synthetic antigen-presenting matrix (in the presence or absence of co-stimulatory molecule(s)); a ligand of the invention covalently or non-covalently complexed to another molecule(s) or macromolecular structure; and an educated, antigen-specific immune effector cell which is specific for a ligand of the invention.

The term “modulate an immune response” includes inducing (increasing, eliciting) an immune response; and reducing (suppressing) an immune response. An immunomodulatory method (or protocol) is one that modulates an immune response in a subject.

As used herein, the term “cytokine” refers to any one of the numerous factors that exert a variety of effects on cells, for example, inducing growth or proliferation. Non-limiting examples of cytokines which may be used alone or in combination in the practice of the present invention include, interleukin-2 (IL-2), stem cell factor (SCF), interleukin 3 colony stimulating factor (GM-CSF), interleukin-1 alpha (IL-1I), interleukin-11 (IL-11), MIP-11, leukemia inhibitory factor (LIF), c-kit ligand, thrombopoietin (TPO) and flt3 ligand. The present invention also includes culture conditions in which one or more cytokine is specifically excluded from the medium. Cytokines are commercially available from several vendors such as, for example, Genzyme (Framingham, Mass.), Genetech (South San Francisco, Calif.), Amgen (Thousand Oaks, Calif.), R&D Systems (Minneapolis, Minn.) and Immunex (Seattle, Wash.). It is intended, although not always explicitly stated, that molecules having similar biological activity as wild-type or purified cytokines (e.g., recombinantly produced or muteins thereof) are intended to be used within the spirit and scope of the invention.

The term “about” or “approximately” will be known to those skilled in the art in light of this disclosure. Preferably, the term means within 20%, more preferably within 10%, and more preferably still within 5% of a given value or range. Alternatively, especially in biological systems, the term “about” preferably means within about a log (i.e., an order of magnitude), preferably within a factor of two of a given value, depending on how quantitative the measurement.

The term “isolated” means separated from constituents, cellular and otherwise, in which the polynucleotide, peptide, polypeptide, protein, antibody, or fragments thereof, are normally associated with in nature. For example, with respect to a polynucleotide, an isolated polynucleotide is one that is separated from the 5′ and 3′ sequences with which it is normally associated in the chromosome. As is apparent to those of skill in the art, a non-naturally occurring polynucleotide, peptide, polypeptide, protein, antibody, or fragments thereof, does not require “isolation” to distinguish it from its naturally occurring counterpart. In addition, a “concentrated”, “separated” or “diluted” polynucleotide, peptide, polypeptide, protein, antibody, or fragments thereof, is distinguishable from its naturally occurring counterpart in that the concentration or number of molecules per volume is greater than “concentrated” or less than “separated” than that of its naturally occurring counterpart. A polynucleotide, peptide, polypeptide, protein, antibody, or fragments thereof, which differs from the naturally occurring counterpart in its primary sequence or for example, by its glycosylation pattern, need not be present in its isolated form since it is distinguishable from its naturally occurring counterpart by its primary sequence, or alternatively, by another characteristic such as glycosylation pattern. Although not explicitly stated for each of the inventions disclosed herein, it is to be understood that all of the above embodiments for each of the compositions disclosed below and under the appropriate conditions, are provided by this invention. Thus, a non-naturally occurring polynucelotide is provided as a separate embodiment from the isolated naturally occurring polynucelotide. A protein produced in a bacterial cell is provided as a separate embodiment from the naturally occurring protein isolated from a eucaryotic cell in which it is produced in nature.

A “subject” is a vertebrate, preferably a mammal, more preferably a human. Mammals include, but are not limited to, murines, simians, humans, farm animals, sport animals, and pets.

A “control” is an alternative subject or sample used in an experiment for comparison purpose. A control can be “positive” or “negative”. For example, where the purpose of the experiment is to determine a correlation of an altered expression level of a gene with a particular type of cancer, it is generally preferable to use a positive control (a subject or a sample from a subject, carrying such alternation and exhibiting syndromes characteristic of that disease), and a negative control (a subject or a sample from a subject lacking the altered expression and clinical syndrome of that disease).

### Molecular Biology—Definitions

A “coding sequence” or a sequence “encoding” an expression product, such as a RNA, polypeptide, protein, or enzyme, is a nucleotide sequence that, when expressed, results in the production of that RNA, polypeptide, protein, or enzyme, i.e., the nucleotide sequence encodes an amino acid sequence for that polypeptide, protein or enzyme. A coding sequence for a protein may include a start codon (usually ATG) and a stop codon.

The term “gene”, also called a “structural gene” means a DNA sequence that codes for or corresponds to a particular sequence of amino acids which comprise all or part of one or more proteins, and may or may not include regulatory DNA sequences, such as promoter sequences, that determine for example the conditions under which the gene is expressed. The transcribed region of a gene can include 5′- and 3′-untranslated regions (UITs) and introns in addition to the translated (coding) region.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

A coding sequence is “under the control” of or “operably (or operatively) associated with” transcriptional and translational control sequences in a cell. RNA polymerase transcribes the coding sequence into mRNA, which is then trans-RNA spliced (if it contains introns) and translated into the protein encoded by the coding sequence. “Operatively associated” refers to a juxtaposition wherein the elements are in an arrangement allowing them to function in concert.

A “targeting sequence” is an amino acid sequence that is tailored to reach a certain specific region of the cytoplasm and/or proteosomes. Such targeting sequences will target proteolysis of the epitopes contained within the constructs of the invention to release the sequences at their intended targets.

The terms “express” and “expression” mean allowing or causing the information in a gene or DNA sequence to become manifest, for example producing a protein by activating the cellular functions involved in transcription and translation of a corresponding gene or DNA sequence. A DNA sequence is expressed in or by a cell to form an “expression product” such as a mRNA or a protein. The expression product itself, e.g. the resulting mRNA or protein, may also be said to be “expressed” by the cell. An expression product can be characterized as intracellular, extracellular or secreted. The term “intracellular” means something that is inside a cell. The term “extracellular” means something that is outside a cell. A substance is “secreted” by a cell if it appears in significant measure outside the cell, from somewhere on or inside the cell. “Conditions that permit expression” in vitro are culture conditions of temperature (generally about 37° C.), humidity (humid atmosphere), carbon dioxide concentration to maintain pH (generally about 5% CO₂ to about 15% CO₂), pH (generally about 7.0 to 8.0, preferably 7.5), and culture fluid components that depend on host cell type. In vivo, the conditions that permit expression are primarily the health of the non-human transgenic animal, which depends on adequate nutrition, water, habitation, and environmental conditions (light-dark cycle, temperature, humidity, noise level). In either system, expression may depend on a repressor or inducer control system, as well known in the art. The term “gene delivery”, “gene transfer”, “transfection”, and the like as used herein mean the introduction of a “foreign” (ie., extrinsic or extracellular) gene, DNA or RNA sequence into a host cell, so that the host cell will express the introduced gene or sequence to produce a desired substance, typically a protein or enzyme encoded by the introduced gene or sequence. The introduced gene or sequence may also be called a “cloned” or “foreign” gene or sequence, may include regulatory or control sequences, such as start, stop, promoter, signal, secretion, or other sequences used by a cell's genetic machinery. The gene or sequence may include nonfunctional sequences or sequences with no known function. A host cell that receives and expresses introduced DNA or RNA has been “transfected” and is a “transfectant” or a “clone.” The DNA or RNA introduced to a host cell can come from any source, including cells of the same genus or species as the host cell, or cells of a different genus or species.

A “gene delivery vehicle” is defined as any molecule that can carry inserted polynucleotides into a host cell. Examples of gene delivery vehicles are liposomes, biocompatible polymers, including natural polymers and synthetic polymers; lipoproteins; polypeptides; polysaccharides; lipopolysaccharides; artificial viral envelopes; metal particles; and bacteria, viruses, such as baculovirus, adenovirus, retrovirus, lentivirus, and adeno-associated virus, bacteriophage, cosmid, plasmid, fungal vectors and other recombination vehicles typically used in the art, which have been described for expression in a variety of eukaryotic and prokaryotic hosts, and may be used for gene therapy as well as for simple protein expression.

The terms “vector”, “cloning vector” and “expression vector” mean the gene delivery vehicle by which a DNA or RNA sequence (e.g., a foreign gene) can be introduced into a host cell, so as to transform the host and promote expression (e.g., transcription and translation) of the introduced sequence.

Vectors typically comprise the DNA of a transmissible agent, into which foreign DNA is inserted. A common way to insert one segment of DNA into another segment of DNA involves the use of enzymes called restriction enzymes that cleave DNA at specific sites (specific groups of nucleotides) called restriction sites. A “cassette” refers to a DNA segment that can be inserted into a vector or into another piece of DNA at a defined restriction site. Preferably, a cassette is an “expression cassette” in which the DNA is a coding sequence or segment of DNA that codes for an expression product that can be inserted into a vector at defined restriction sites. The cassette restriction sites generally are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct.” A common type of vector is a “plasmid” that generally is a self-contained molecule of double-stranded DNA, usually of bacterial origin, that can readily accept additional (foreign) DNA and which can be readily introduced into a suitable host cell. A plasmid vector often contains coding DNA and promoter DNA and has one or more restriction sites suitable for inserting foreign DNA. A large number of vectors, including plasmid and fungal vectors, have been described for replication and/or expression in a variety of eukaryotic and prokaryotic hosts. Non-limiting examples include pKK plasmids (Amersham Pharmacia Biotech), pUC plasmids, pET plasmids (Novagen, Inc., Madison, Wis.), pRSET or pREP plasmids (Invitrogen, San Diego, Calif.), or pMAL plasmids (New England Biolabs, Beverly, Mass.), and many appropriate host cells, using methods disclosed or cited herein or otherwise known to those skilled in the relevant art. Recombinant cloning vectors will often include one or more replication systems for cloning or expression, one or more markers for selection in the host, e.g. antibiotic resistance, and one or more expression cassettes.

The term “host cell” or “recipient cell” means any cell of any organism that is selected, modified, transformed, grown, or used or manipulated in any way by introduction of a polynucleotide or vector encoding a variant construct, for the production of a substance by the cell, for example the expression by the cell of a gene, a DNA or RNA sequence, or protein, i.e., the variant construct. The host cell may be found in vitro, i.e., in tissue culture, or in vivo, i.e., in a microbe, plant or animal. These terms are intended to include progeny of a single cell, and progeny may not necessarily be completely identical (in morphology or in genomic or total DNA complement) to the original parent cell due to natural, accidental, or deliberate mutation or introduction of further variations. The cells may be prokaryotic or eukaryotic, and include, but are not limited to, bacterial cells, yeast cells, animal cells, and mammalian cells, e.g., murine, rat, hamster, simian, or human cells.

The term “expression system” means a host cell and compatible vector under suitable conditions, e.g., for the expression of a protein coded for by foreign DNA carried by the vector and introduced to the host cell. Preferably the introduced polynucleotide is stably or transiently maintained in the host cell. Stable maintenance typically requires that the introduced polynucleotide either contains an origin of replication compatible with the host cell or integrates into a replicon of the host cell such as an extrachromosoml replicon (e.g. a plasmid) or a nuclear or mitochondrial chromosome. Common expression systems include E. coli host cells and plasmid vectors, insect host cells and Baculovirus vectors, and mammalian host cells and vectors. In a specific embodiment, the construct is expressed in COS-1 or CHO cells. Other suitable cells include NSO cells, HeLa cells, 293T (human kidney cells), mouse primary myoblasts and NIH 3T3 cells.

The term “culturing” refers to the in vitro propagation of host cells or organisms on or in media of various kinds. Preferably, culturing occurs under conditions that permit expression of the variant construct. By “expanded” is meant any proliferation or division of cells.

The terms “mutant” and “mutation” mean any detectable change in genetic material, e.g., DNA, or any process, mechanism, or result of such a change. This includes gene mutations, in which the structure (e.g., DNA sequence) of a gene is altered, any gene or DNA arising from any mutation process, and any expression product (e.g. protein or enzyme) expressed by a modified gene or DNA sequence. The term “variant” may also be used to indicate a modified or altered gene, DNA sequence, enzyme, cell, etc., i.e., any kind of mutant.

“Sequence-conservative variants” of a polynucleotide sequence are those in which a change of one or more nucleotides in a given codon position results in no alteration in the amino acid encoded at that position.

“Function-conservative variants” are those in which a given amino acid residue in a protein or enzyme has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids with similar properties are well known in the art. For example, arginine, histidine and lysine are hydrophilic-basic amino acids and may be interchangeable. Similarly, isoleucine, a hydrophobic amino acid, may be replaced with leucine, methionine or valine. Such changes are expected to have little or no effect on the apparent molecular weight or isoelectric point of the protein or polypeptide. Amino acids other than those indicated as conserved may differ in a protein or enzyme so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A “function-conservative variant” also includes a polypeptide or enzyme which has at least 60% amino acid identity as determined by BLAST (including BLASTN, BLASTP, and BLASTX) or FASTA algorithms, preferably at least 75%, more preferably at least 85%, and even more preferably at least 90%, and which has the same or similar properties or functions as the native or parent protein or enzyme to which it is compared.

A polynucleotide or polynucleotide region (or polypeptide or polypeptide region) has a certain percentage (for example 80%, 85%, 90%, or 95%) of “sequence identity” to another sequence if the percentage of bases (or arnino acids) are the same when the two sequences are aligned and compared. The alignment and percent homology or sequence identigy can be determined using software programs described above (see Ausubel et al., Current Protocols In Molecular Biology, 1987, Supplement 30, section 7.7.18, Table 7.7.1). Preferably default parameters are sued for alignment. For BLAST programs, the default parameters preferably are: Genetic code=standard; filter=none; strand=both; cutoff=60; expect=10; Matrix=BLOSUM62; Descriptions=50 sequences; sort by=HIGH SCORE; databases=non-redundant GenBank+EMBL+DDBJ+PDB+GenBank CDS translations+SwissProtein+Spupdate+PIR.

As used herein, the term “oligonucleotide” refers to a nucleic acid, generally of at least 10, preferably at least 15, and more preferably at least 20 nucleotides, preferably no more than about 100 nucleotides, that is hybridizable to a genomic DNA molecule, a cDNA molecule, or an mRNA molecule having a sequence of interest. Oligonucleotides can be labeled, e.g., with ³²P-nucleotides or nucleotides to which a label, such as biotin, has been covalently conjugated. In one embodiment, a labeled oligonucleotide can be used as a probe to detect the presence of a nucleic acid. In another embodiment, oligonucleotides (one or both of which may be labeled) can be used as PCR primers, either for cloning full length or a fragment of the synthebody, or to detect the presence of nucleic acids encoding the synthebody. In a further embodiment, an oligonucleotide of the invention can form a triple helix with a synthebody-encoding DNA molecule, e.g., for purification purposes. Generally, oligonucleotides are prepared synthetically, preferably on a nucleic acid synthesizer. Accordingly, oligonucleotides can be prepared with non-naturally occurring phosphoester analog bonds, such as thioester bonds, etc.

### Constructs

The constructs of the invention can be derived from any type of immunoglobulin molecule, for example, but not limited to, antibodies, T lymphocyte receptors, cell-surface adhesion molecules such as the co-receptors CD4, CD8, CD19, and the invariant domains of MHC molecules. In a preferred embodiment of the invention, the construct is derived from an antibody, which can be any class of antibody, e.g., an IgG, IgE, IgM, IgD or IgA, preferably, the antibody is an IgG. Such antibodies may be in membrane bound (B cell receptor) or secreted form, preferably secreted. Additionally, the antibody may be of any subclass of the particular class of antibodies. In another specific embodiment, the construct is derived from a T lymphocyte receptor.

In one embodiment, the invention provides antibodies that are specific for p53BP2 ligand polypeptides identified as described above. The antibodies are polyclonal or monoclonal, and discriminate p53BP2 ligand from different species, identify functional domains, and the like. Such antibodies are conveniently made using the methods and compositions disclosed in Harlow and Lane, Antibodies, A Laboratory Manual, Cold Spring Harbor Laboratory, 1988, as well as immunological and hybridoma technologies known to those skilled in the art. Where natural or synthetic p53BP2 ligand peptides are used to induce an p53BP2 ligand specific immune response, the peptides are conveniently coupled to a suitable carrier such as KLH and administered in a suitable adjuvant such as Freund's. Preferably, selected peptides are coupled to a lysine core carrier substantially according to the methods of Tan (1988) Proc. Natl. Acad. Sci. USA, 85: 5049-5413. The resulting antibodies, especially internal imaging anti-idiotypic antibodies, are also prepared using known methods.

In one embodiment, purified p53BP2 ligands are used to immunize mice, after which their spleens are removed, and splenocytes used to form cell hybrids with myeloma cells to obtain clones of antibody-secreting cells according to techniques that are standard in the art. The resulting monoclonal antibodies secreted by such cells are screened using in vitro assays for biological activity, including but not limited to, binding to p53 receptor.

Alternatively, the invention provides antibodies having CDR-grafted variable regions. CDR-grafted variable region genes have been constructed by various methods such as site-directed mutagenesis as described in Jones et al., Nature 1986, 321:522; Riechmann et al., Nature 1988, 332:323; in vitro assembly of entire CDR-grafted variable regions (Queen et al., Proc. Natl. Acad. Sci. USA 1989, 86:10029); and the use of PCR to synthesize CDR-grafted genes (Daugherty et al., Nucleic Acids Res. 1991, 19:2471). CDR-grafted antibodies are generated in which the CDRs of the murine monoclonal antibody are grafted onto the framework regions of a human antibody. Following grafting, most antibodies benefit from additional amino acid changes in the framework region to maintain affinity, presumably because framework residues are necessary to maintain CDR conformation, and some framework residues have been demonstrated to be part of the antigen combining site. Such CDR-grafted antibodies have been successfully constructed against various antigens, for example, antibodies against IL-2 receptor as described in Queen et al. (Proc. Natl. Acad. Sci. USA 1989, 86:10029), antibodies against cell surface receptors-CAMPATH as described in Riechmann et al. (Nature, 1988, 332:323); antibodies against hepatitis B in Co et al. (Proc. Natl. Acad. Sci. USA 1991, 88:2869); as well as against viral antigens of the respiratory syncitial virus in Tempest et al. (BioTechnology 1991, 9:267). Thus, in specific embodiments of the invention, the construct comprises a variable domain in which at least one of the framework regions has one or more amino acid residues that differ from the residue at that position in the naturally occurring framework region. The techniques employed in creating CDR-grafted antibodies can be adapted for use in constructs of the invention.

The heterologous amino acid sequence of sequences can be inserted into any one or more of the CDR regions of the variable domain variant, preferably into the corresponding CDR or CDRs of the variable domain variant. It is within the skill in the art to insert sequences from the ligand having immunogenic properties relevant to human lung cancer into different or corresponding CDRs of the variable domain or domains of the synthetic construct, and then screen the resulting modified constructs for the ability to elicit an appropriate anti-idiotypic antibody response. In specific embodiments in which the construct is an antibody, a CDR or CDRs of either the heavy or light chain variable region, or both, are modified to contain the heterologous amino acid sequence. In another specific embodiment, the construct contains a variable domain in which the first, second, and third CDR of the heavy chain variable region or the first, second, and third CDR of the light chain variable region, and preferably both, contain the antigenic amino acid sequence of the ligand. Optionally, the first, second, and third CDR of the heavy chain variable region and/or the first, second, and third CDR of the light chain variable region also contains one or more of the following: (a) CTL epitope sequence; (b) T-helper cell sequence; (c) a B-helper cell sequence; (d) a targeting sequence, or combinations thereof. Corresponding modifications are also contemplated for other immunoglobulin superfamily protein-derived constructs of the invention.

In specific embodiments of the invention, the heterologous amino acid sequence(s) is either inserted into the CDR without replacing any of the amino acid sequence of the CDR itself or, alternatively, the binding site amino acid sequence replaces all or a portion of the amino acid sequence of the CDR.

Relative efficacy of a construct to generate an anti-tumor anti-idiotype response can be evaluated by direct binding assays, such as ELISA, Western blotting, and the like; and functional assays, including tumor clearance in in vivo animal models of lung cancer.

The constructs of the invention may also be further modified in any way known in the art, e.g., for the modification of antibodies as long as the further modification does not completely prevent binding of the construct to the particular binding partner. In particular, the constructs of the invention may have one or more amino acid substitutions, deletions, or insertions besides the insertion into or replacement of CDR sequences with the binding sequence. Such amino acid substitutions, deletions, or insertions can be any substitution, deletion, or insertion that does not prevent the specific binding of the construct to the target binding partner. For example, such amino acid substitutions include substitutions of functionally equivalent amino acid residues. One or more amino acid residues can be substituted by another amino acid of a similar polarity that acts as a functional equivalent resulting in a silent alteration. Substitutes for an amino acid may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid.

Additionally, one or more amino acid residues can be substituted by a nonclassical amino acid or chemical amino acid analogs, introduced as a substitution or addition into the immunoglobulin sequence. Non-classical amino acids include but are not limited to the D-isomers of the common amino acids, alpha-amino isobutyric acid, 4-aminobutyric acid, 2-aminobutyric acid, 6-amino hexanoic acid, 2-amino isobutyric acid, 3-amino propionic acid, ornithine, norleucine, norvaline, hydroxyproline, sarcosine, cifrulline, cysteic acid, t-butylglycine, t-butylalanine, phenylglycine, cyclohexylalanine, beta-alanine, fluoro-amino acids, designer amino acids such as beta-methyl amino acids, C-alpha-methyl amino acids, N-alpha-methyl amino acids, and amino acid analogs in general. Furthermore, the amino acid can be D (dextrorotary) or L (levorotary).

### Anti-Idiotype Constructs

To ensure a robust anti-idiotype response, the construct is further modified to enhance its ability to elicit an anti-idiotype response, for example, as described in PCT Publication No. WO 99/25379. Such modifications are made to reduce the conformational constraints on a variable domain, e.g., by removing or reducing intrachain disulfide bonds. Specifically, the construct is further modified such that one or more variable region cysteine residues that form disulfide bonds are replaced with an amino acid residue that does not have a sulfhydryl group.

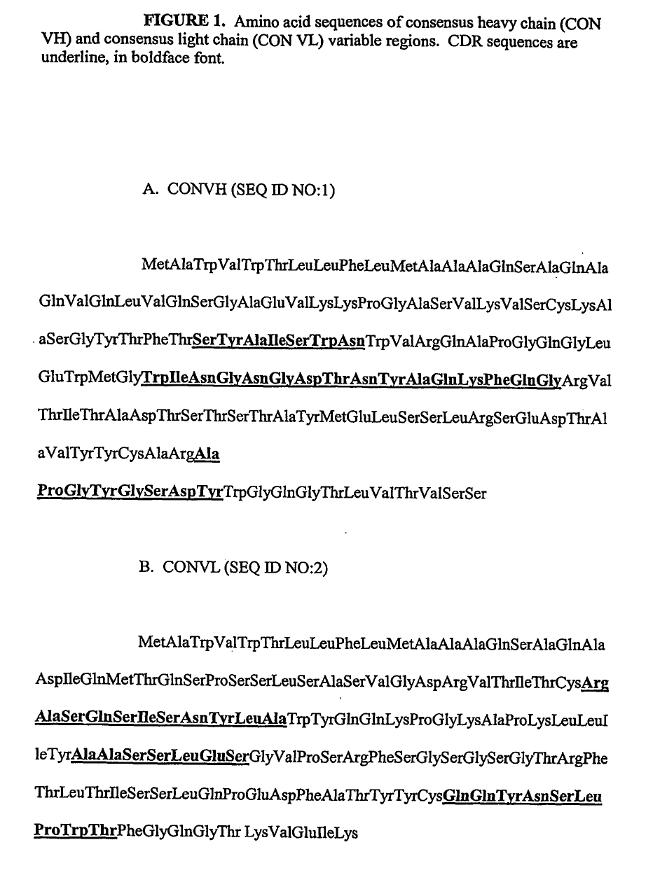

Identifying the cysteine residues that form a disulfide bond in a variable region of a particular antibody can be accomplished by any method known in the art. For example, but not by way of limitation, it is well known in the art that the cysteine residues that form intrachain disulfide bonds are highly conserved among antibody classes and across species. Thus, the cysteine residues that participate in disulfide bond formation can be identified by sequence comparison with other antibody molecules in which it is known which residues form a disulfide bond (for example the consensus sequences provided in FIGS. 1A and 1B, or those described in Kabat et al., 1991, Sequences of Proteins of Immunological Interest, 5^{th} Ed., U.S. Department of Health and Human Services, Bethesda, Md.).

Notably, for most antibody molecules, the cysteine residues that form the intrachain disulfide bonds are residues at positions 23 and 88 of the light chain variable domain and residues at positions 22 and 92 of the heavy chain variable domain. The position numbers refer to the residue corresponding to that residue in the consensus sequences as defined in Kabat et al., supra, i.e. the “Kabat equivalent” or as indicated in the heavy and light chain variable region sequences depicted in FIGS. 1A and 1B, respectively (as determined by aligning the particular antibody sequence with the consensus sequence of the heavy or light chain variable region sequence depicted in FIGS. 1A and 1B).

Accordingly, in one embodiment of the invention, the construct is further modified such that the residues at positions 23 and/or 88 of the light chain as identified by Kabat et al., supra, or their equivalent, are substituted with an amino acid residue that does not contain a sulfhydryl group; and/or the residues at positions 22 and/or 92 are of the heavy chain as identified by Kabat et al., supra, or their equivalent, are substituted with an amino acid residue that does not contain a sulfhydryl group. As used in reference to intrachain disulfides, equivalents of the cysteines at the Kabat positions provided above are intrachain-forming cysteine residues at homologous positions in the immunoglobulin domain of an immunoglobulin superfamily protein molecule.

The amino acid residue that substitutes for the disulfide bond forming cysteine residue is any amino acid residue that does not contain a sulfhydryl group, e.g. alanine, arginine, asparagine, aspartate (or aspartic acid), glutamine, glutamate (or glutamic acid), glycine, histidine, isoleucine, leucine, lysine, phenylalanine, proline, serine, threonine, tryptophan, tyrosine or valine. In a preferred embodiment, the cysteine residue is replaced with a glycine, serine, threonine, tyrosine, asparagine, or glutamine residue, most preferably with an alanine residue.

Additionally, the disulfide bond forming cysteine residue may be replaced by a nonclassical amino acid or chemical amino acid analog, such as those listed-supra, that does not contain a sulfhydryl group; or it may be chemically modified by reaction with the sulfhydryl to preclude disulfide bond formation.

In specific embodiments, the substitution of the disulfide bond forming residue is in the heavy chain variable region or the light chain variable region, or both the heavy chain and light chain variable regions. In other specific embodiments, one of the residues that forms a particular disulfide bond is replaced (or deleted) or, alternatively, both residues that form a particular disulfide bond may be replaced (or deleted).

### Immunoglobulin Fragment Constructs

As noted above, fragments of an immunoglobulin family protein can be modified to create a construct. For example, such fragments include but are not limited to: F(ab′)₂ fragments that contain the variable regions of both the heavy and the light chains, the light constant region and the CH1 domain of the heavy chain, which fragments can be generated by pepsin digestion of an antibody; Fab′ fragments; Fab fragments generated by reducing the disulfide bonds of an F(ab′)₂ fragment (King et al., Biochem. J., 1992, 281:317); and Fv fragments, i.e., fragments that contain the variable region domains of both the heavy and light chains (Reichmann and Winter, J. Mol. Biol. 1988, 203:825; King et al., Biochem J. 1993, 290:723).

The present invention also includes, but is not limited to, single chain antibodies (SCA) (U.S. Pat. No. 4,946,778; Bird, Science 1988, 242:423-426; Huston et al., Proc. Natl Acad. Sci. USA 1988, 85:5879-5883; and Ward et al., Nature 1989, 334:544-546). Single chain antibodies are formed by linking the heavy and light chain fragments of the Fv region via an amino acid bridge, resulting in a single chain polypeptide. Additionally, the invention also provides heavy chain and light chain dimers and diabodies.

### Preferred Immunoglobulin Family Proteins

The immunoglobulin molecule modified to generate the constructs is preferably a monoclonal antibody. The term “antibody” is intended to include antibodies of all isotypes and species. Particular isotypes of monoclonal antibody can be prepared either directly by selecting from the initial fusion, or prepared secondarily, from a parental hybridoma secreting a monoclonal antibody of different isotype by using the sib selection technique to isolate class switch variants (Steplewski et al., Proc. Natl. Acad. Sci USA 1985, 82:8653; Spira et al., J. Immunol. Meth. 1984, 74:307). The antibody that is modified may be a naturally occurring or previously existing antibody, or may be synthesized from known antibody consensus sequences, such as the consensus sequences for the light and heavy chain variable regions in FIGS. 1A and 1B, or any other antibody consensus or germline (i.e., unrecombined genomic sequences) sequences (e.g., those antibody consensus and germline sequences described in Kabat et al., 1991, Sequences of Proteins of Immunological Interest, 5^{th} edition, NIH Publication No. 91-3242, pp. 2147-2172).

The invention further provides constructs that are modified chimeric or humanized antibodies. A chimeric antibody is a molecule in which different portions of the antibody molecule are derived from different animal species, such as those having a variable region derived from a murine mAb and a constant region derived from a human immunoglobulin constant region. Techniques have been developed for the production of chimeric antibodies (Morrison et al., Proc. Natl. Acad. Sci. USA 1984, 81:6851-6855; Neuberger et al., Nature, 1984, 312:604-608; Takeda et al., Nature 1985, 314:452-454; Oi et al., BioTechniques 1986, 4:214; International Patent Application No. PCT/GB85/00392) by splicing the genes from a mouse antibody molecule of appropriate antigen specificity together with genes from a human antibody molecule of appropriate biological activity. In a specific embodiment, the synthebody is a chimeric antibody containing the variable domain of a non-human antibody and the constant domain of a human antibody.

In another embodiment, the construct is derived from a humanized antibody, in which the CDRs of the antibody (except for the one or more CDRs containing the heterologous binding sequence) are derived from an antibody of a non-human animal and the framework regions and constant region are from a human antibody (see, U.S. Pat. No. 5,225,539; Oi et al., supra).

As noted above, the construct can be derived from a human monoclonal antibody. The creation of completely human monoclonal antibodies is possible through the use of transgenic mice. Transgenic mice in which the mouse immunoglobulin gene loci have been replaced with human immunoglobulin loci provide in vivo affinity-maturation machinery for the production of human immunoglobulins.

### Immunoglobulin Fusion Protein and Derivative Construct

In certain embodiments, the construct is created by fusing (joining) an immunoglobulin family protein modified to include the heterologous binding sequence to an amino acid sequence of another protein (or portion thereof, preferably an at least 10, 20, or 50 amino acid portion thereof) that is not the modified immunoglobulin, thereby creating a fusion (or chimeric) construct. Preferably, the fusion is via covalent bond (for example, but not by way of limitation, a peptide bond) at either the N-terminus or the C-terminus.

The construct may be further modified, e.g., by the covalent attachment of any type of molecule, as long as such covalent attachment does not prevent or inhibit anti-tumor anti-idiotype respose. For example, but not by way of limitation, the construct may be further modified, e.g., by glycosylation, acetylation, PEGylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formylation, metabolic synthesis of tunicamycin, etc.

In specific embodiments of the invention, the construct is covalently linked to a therapeutic molecule, for example, to target the therapeutic molecule to a particular cell type or tissue, e.g., an accessory or antigen-presenting cell. The therapeutic molecule can be any type of therapeutic molecule known in the art, for example, but not limited to, a chemotherapeutic agent, a toxin, such as ricin, an antisense oligonucleotide, a radionuclide, an antibiotic, anti-viral, or anti-parasitic, etc.

### Ligands and Antigenic Sequences

The present invention provides novel ligands, compounds and related compositions for therapeutic, prophylactic and diagnostic use. In a first aspect, this invention provides a novel ligand and corresponding protein, neither of which were heretofore known to be associated with p53 (Naumovski, L. and Cleary, M. L. Mol. Cell Bio. 16(7):3884-3892 (1996)). In a different aspect, this invention provides novel sequences, i.e., antigenic ligand sequences that are “cross-reactive” with the native ligand (“altered ligands”).

The native and the altered ligands of the invention are useful in therapeutic, prophylactic, and diagnostic applications by incorporating their antigenic sequences in constructs.

The ligand sequence containing constructs of the invention are useful as immunogens to generate antibodies that specifically recognize and bind to these ligands. The antibodies so generated are further useful in therapeutic and diagnostic applications.

The present invention provides compounds having the following structures:

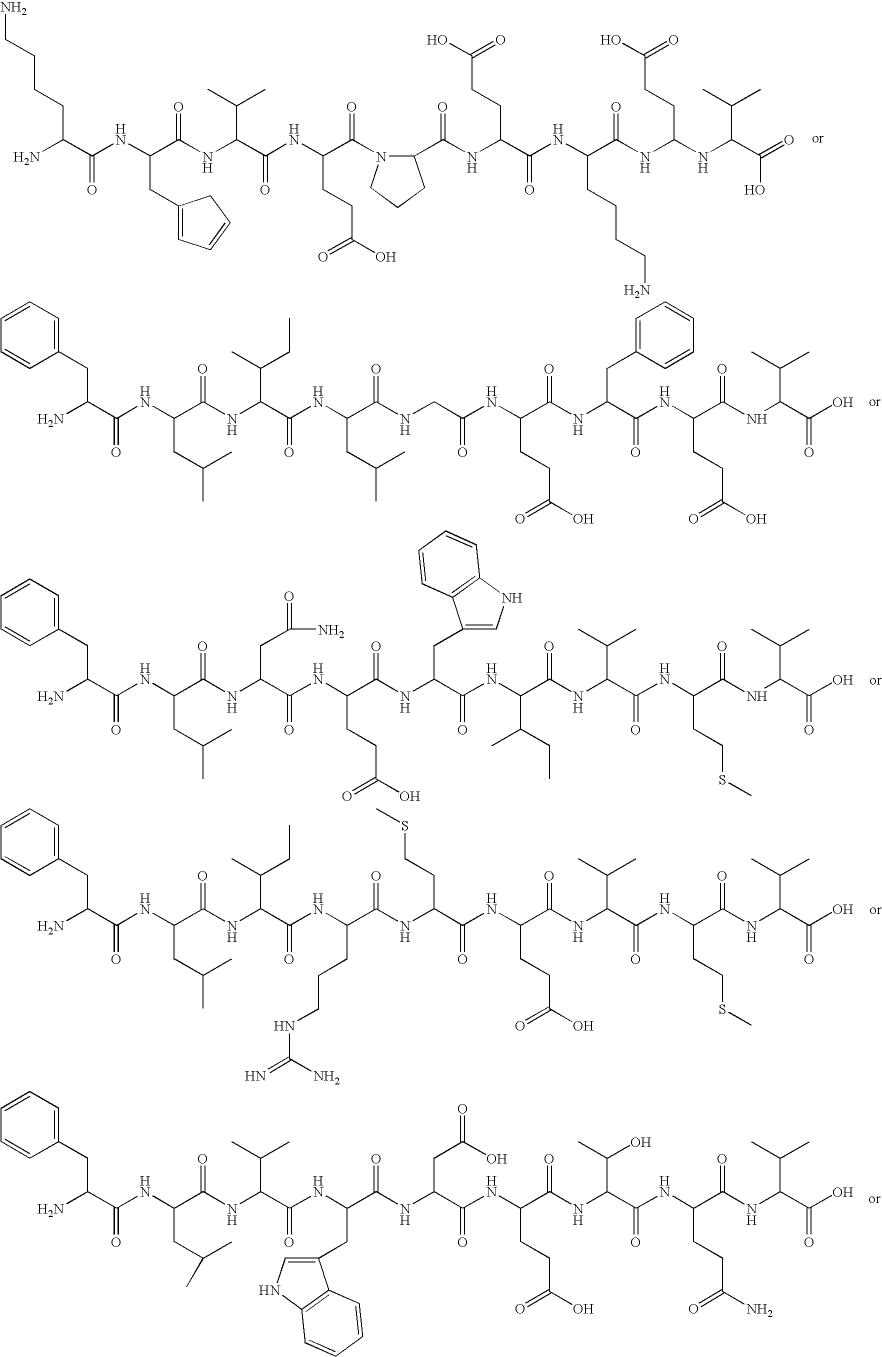

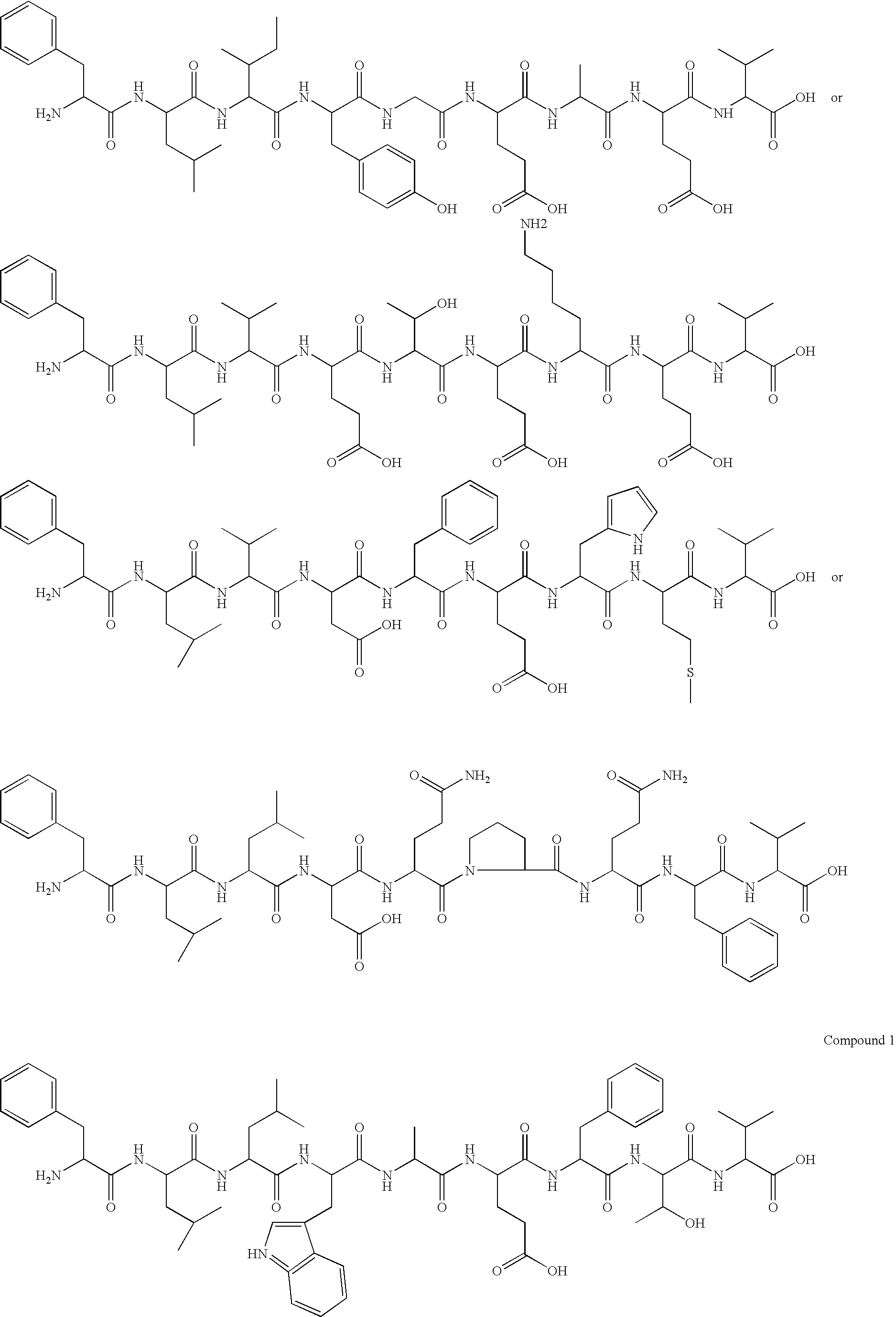

This invention further provides compositions which are useful as components of anti-cancer vaccines and to expand immune effector cells that are specific for cancers characterized by expression p53BP2 ligand.

In one embodiment, the altered ligands of the invention have comparable affinity for MHC binding as the native ligand. It has been demonstrated that peptide:MHC class I binding properties correlate with immunogenicity (Setts, A. et al, Immunol. 1994, 153:5586; van der Burg, S. H. et al, J. Immunol. 1996, 156:3308). In a preferred embodiment, altered ligands of the invention bind to a TCR with a higher affinity than of that the “natural ligand. Comparative binding of the native and altered ligands of the invention to an MHC class I molecule can be measured by methods that are known in the art and include, but are not limited to, calculating the affinity based on an algorithm (see, for example, Parker et al, J. Immunol. 1992, 149:3580-3587); and experimentally determining binding affinity (see, for example, Tan et al, J. Immunol. Metho. 1997, 209:25-36. For example, the relative binding of a peptide to a class I molecule can be measured on the basis of binding of a radiolabeled standard peptide to detergent-solubilized MHC molecules, using various concentrations of test peptides (e.g., ranging from 100 mM to 1 nM). MHC class I heavy chain and β2-microglobulin are coincubated with a fixed concentration (e.g., 5 nM) radiolabeled standard (control) peptide and various concentrations of a test peptide for a suitable period of time (e.g., 2 hours to 72 hours) at room temperature in the presence of a mixture of protease inhibitors. A control tube contains standard peptide and MHC molecules, but no test peptide. The percent MHC-bound radioactivity is determined by gel filtration. The IC₅₀ (concentration of test peptide which results in 50% inhibition of binding of control peptide) is calculated for each peptide. Additional methods for determining binding affinity to a TCR are known in the art (see e.g., al-Ramadi et al, J. Immunol 1992, 155(2):662-673; and Zuegel et al, J. Immunol. 1998, 161(4):1705-1709).

In another embodiment, the altered ligands of the invention elicit comparable antigen-specific T cell activation relative to their native ligand counterpart. In a preferred embodiment, altered ligands of the invention elicit a stronger antigen-specific T cell activation relative to their native ligand counterpart. Methods for determining immunogenicity of invention ligands are known in the art and are further described herein.

In one embodiment, compositions of the invention comprise two or more antigenic sequences from the immunogenic ligand of the invention. In one aspect, such composition may comprise two or more copies of a single ligand sequence. In another aspect, such compositions may comprise two or more different sequences from the ligand, or more than one ligand, wherein each sequence of the one or more ligands is distanct from other sequences.

### Structure of the Heterologous Antigenic Lizand (Amino Acid) Sequence

One or more, including, all six CDRs contained in the consensus heavy and light chain variable region clones with a sequence or sequences from ligands having SEQ ID NOS. 3, 5, 7, 9, 11, 13, 15, 17, 19 or 21.

### Methods of Producing the Constructs

Constructs can be produced by any method known in the art for the synthesis of immunoglobulins, in particular, by chemical synthesis or by recombinant expression, and are preferably produced by recombinant expression techniques.

Recombinant expression of constructs requires construction of a nucleic acid encoding the construct. Such an isolated nucleic acid that contains a nucleotide sequence encoding the construct can be produced using any method known in the art.

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning.: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization, B. D. Hames & S. J. Higgins eds. (1985); Transcription And Translation, B. D. Hames & S. J. Higgins, eds. (1984); Animal Cell Culture, R. I. Freshney, ed. (1986); Immobilized Cells And Enzymes, IRL Press, (1986); B. Perbal, A Practical Guide To Molecular Cloning (1984); F. M. Ausubel et al. (eds.), Current Protocols in Molecular Biology, John Wiley & Sons, Inc. (1994); Goeddel et al., Gene Expression Technology, Academic Press (1991); Gacesa and Ramji, Vectors: Essential Data Series, John Wiley & Sons (1994).

### Construct Nucleic Acids

Accordingly, the invention provides nucleic acids that contain a nucleotide sequence encoding a construct of the invention.

A nucleic acid that encodes a construct may be assembled from chemically synthesized oligonucleotides (e.g., as described in Kutmeier et al., BioTechniques 1994, 17:242), that briefly, involves the synthesis of a set of overlapping oligonucleotides containing portions of the sequence encoding the protein, annealing and ligation of those oligonucleotides, and then amplification of the ligated oligonucleotides by PCR.

RNA can be obtained by first inserting a DNA polynucleotide into a suitable host cell. The DNA can be inserted by an appropriate method, as described above. When the cell replicates and the DNA is transcribed into RNA, the RNA can be isolated using methods well known to those of skill in the art, for example, as set forth in Sambrook, supra. For instance, mRNA can be isolated using various lytic enzymes or chemical solutions according to the procedures in Sambrook, supra, or extracted by nucleic acid-binding resins following the instructions provided by the resin manufacturers.

Accordingly, the invention provides a method of producing a nucleic acid encoding a construct, the method comprising: (a) synthesizing a set of oligonucleotides, the set comprising oligonucleotides containing a portion of the nucleotide sequence that encodes the construct and oligonucleotides containing a portion of the nucleotide sequence that is complementary to the nucleotide sequence that encodes the construct, and each of the oligonucleotides having overlapping terminal sequences with another oligonucleotide of the set, except for those oligonucleotides containing the nucleotide sequences encoding the N-terminal and C-terminal portions of the synthetic synthebody; (b) allowing the oligonucleotides to hybridize or anneal to each other; and (c) ligating the hybridized oligonucleotides, such that a nucleic acid containing the nucleotide sequence encoding the synthetic synthebody is produced.

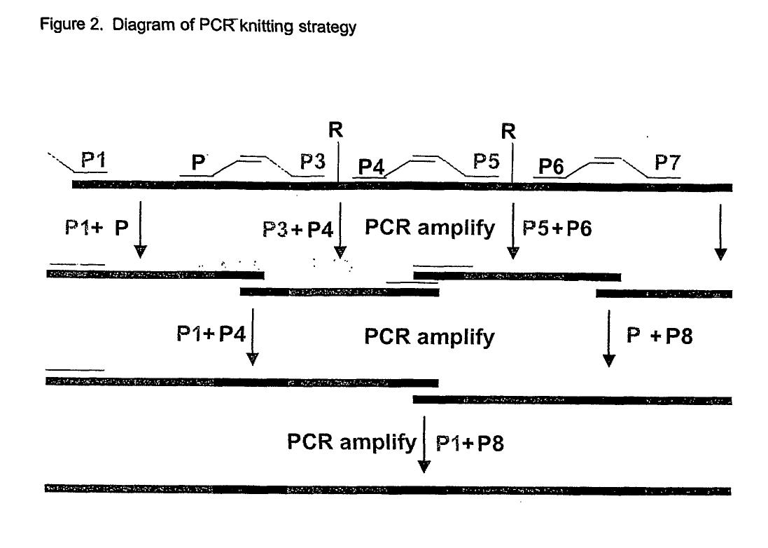

Another method for producing a nucleic acid encoding a construct is to modify nucleic acid sequences that encode an immunoglobulin superfamily molecule, e.g., an antibody molecule or at least the variable region thereof, using the “PCR knitting” approach FIG. 2). In “PCR knitting”, nucleic acid sequences, such as the consensus variable region sequences shown in Example 1, are used as templates for a series of PCR reactions that result in the selective insertion of a nucleotide sequence that encodes the desired peptide sequence (in this example, the antigenic sequences of the ligand p53BP2) into one or more CDRs of the variable domain. Oligonucleotide primers are designed for these PCR reactions that contain regions complementary to the framework sequences flanking the designated CDR at the 3′ends and sequences that encode the peptide sequence to be inserted at the 5′ends. In addition, these oligonucleotides contain approximately ten bases of complementary sequences at their 5′ends. These oligonucleotide primers can be used with additional flanking primers to insert the desired nucleotide sequence into the selected CDR as shown in FIG. 2 resulting in the production of a nucleic acid coding for the synthebody.

Alternatively, a nucleic acid containing a nucleotide sequence encoding a construct can be constructed from a nucleic acid containing a nucleotide sequence encoding, e.g., an antibody molecule, or at least a variable region of an antibody molecule. Nucleic acids containing nucleotide sequences encoding antibody molecules can be obtained either from existing clones of antibody molecules or variable domains or by isolating a nucleic acid encoding an antibody molecule or variable domain from a suitable source, preferably a cDNA library, e.g., an antibody DNA library or a cDNA library prepared from cells or tissue expressing a repertoire of antibody molecules or a synthetic antibody library (see, e.g., Clackson et al., Nature, 1991, 352:624; Hane et al., Proc. Natl. Acad. Sci. USA, 1997, 94:4937), for example, by hybridization using a probe specific for the particular antibody molecule or by PCR amplification using synthetic primers hybridizable to the 3′ and 5′ ends of the sequence.

If a convenient restriction enzyme site is available in the nucleotide sequence of the CDR, then the sequence can be cleaved with the restriction enzyme and a nucleic acid fragment containing the nucleotide sequence encoding the binding site can be ligated into the restriction site. The nucleic acid fragment containing the binding site can be obtained either from a nucleic acid encoding all or a portion of the protein containing the binding site or can be generated from synthetic oligonucleotides containing the sequence encoding the binding site and its reverse complement.

The nucleic acid encoding the modified antibody optionally contains a nucleotide sequence encoding a leader sequence that directs the secretion of the synthebody molecule.

### Construct Expression

Once a nucleic acid encoding a construct is obtained, it may be expressed or it may be introduced into a vector containing the nucleotide sequence encoding the constant region of the antibody (see, e.g., PCT Publications WO 86/05807 and WO 89/01036; and U.S. Pat. No. 5,122,464). Vectors containing the complete light or heavy chain for co-expression are available to allow the expression of a complete antibody molecule and are known in the art, for example, pMRO10.1 and pGamma1 (see also, Bebbington, Methods a companion to Methods in Enzymology 1991, 2:136-145).

The expression vector can then be transferred to a host cell in vitro or in vivo by conventional techniques and the transfected cells can be cultured by conventional techniques to produce a construct of the invention. Specifically, once a variable region of the modified antibody has been generated, the modified antibody can be expressed, for example, by the method exemplified in the Examples (see also Bebbington, supra). For example, by transient transfection of the expression vector encoding a construct into COS cells, culturing the cells for an appropriate period of time to permit construct expression, and then taking the supernatant from the COS cells, which supernatant contains the secreted, expressed synthebody.

The host cells used to express the recombinant construct of the invention may be either bacterial cells such as Escherichia coli, particularly for the expression of recombinant antibody fragments or, preferably, eukaryotic cells, particularly for the expression of recombinant immunoglobulin molecules. In particular, mammalian cells such as Chinese hamster ovary cells (CHO) or COS cells, used in conjunction with a vector in which expression of the construct is under control of the major intermediate early gene promoter element from human cytomegalovirus, is an effective expression system for immunoglobulins (Foecking et al., Gene 1986, 45:101; Cockett et al., BioTechnology 1990, 8:662).

A variety of host-expression vector systems may be utilized to express the construct coding sequences of the invention. Such host-expression systems represent vehicles by which the coding sequences of interest may be produced and subsequently purified, but may also be used to transform or transfect cells with the appropriate nucleotide coding and control sequences to produce the antibody product of the invention in situ. These systems include, but are not limited to, microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing the antibody coding sequences; plant cell systems infected with recombinant virus expression vectors (e.g., cauliflower mosaic virus, CaMV; tobacco mosaic virus, tMV) or transformed with recombinant plasmid expression vectors (e.g., Ti plasmid) containing antibody coding sequences; mammalian cell systems (e.g., COS, CHO, BHK, 293, and 3T3 cells) harboring recombinant expression constructs containing promoters derived from the genome of mammalian cells (e.g., the metallothionein promoter) or from mammalian viruses (e.g. the adenovirus late promoter; the vaccinia virus 7.5K promoter); and transgenic animal systems, particularly for expression in milk (e.g., U.S. Pat. Nos. 5,831,141 and 5,849,992, which describe transgenic production of antibodies in milk; U.S. Pat. No. 4,873,316).

Expression of the construct may be controlled by any promoter/enhancer element known in the art, but these regulatory elements must be functional in the host selected for expression and operatively associated with the sequence encoding the variant construct. Promoters that may be used to control gene expression include, but are not limited to, cytomegalovirus (CMV) promoter (U.S. Pat. Nos. 5,385,839 and 5,168,062), the SV40 early promoter region (Benoist and Chambon, Nature 1981, 290:304-310), the promoter contained in the 3′ long terminal repeat of Rous sarcoma virus (Yamamoto, et al. Cell, 1980, 22:787-797), the herpes thymidine kinase promoter (Wagner et al., Proc. Natl. Acad. Sci. USA 1981, 78:1441 -1445), the regulatory sequences of the metallothionein gene (Brinster et al., Nature 1982, 296:39-42); prokaryotic expression vectors can use SP6, T4, or T7 promoters, the β-lactamase promoter (Villa-Komaroff, et al., Proc. Natl. Acad. Sci. USA 1978, 75:3727-3731), or the tac promoter (DeBoer, et al., Proc. Natl. Acad. Sci. USA 1983, 80:21-25); see also “Useful proteins from recombinant bacteria” in Scientific American 1980, 242:74-94; promoter elements from yeast or other fungi such as the Gal 4 promoter, the ADC (alcohol dehydrogenase) promoter, PGK (phosphoglycerol kinase) promoter, alkaline phosphatase promoter; and transcriptional control regions that exhibit hematopoietic tissue specificity, in particular: beta-globin gene control region which is active in myeloid cells (Mogram et al., Nature 1985, 315:338-340; Kollias et al. 1986, Cell 46:89-94), hematopoietic stem cell differentiation factor promoters, erythropoietin receptor promoter (Maouche et al., Blood 1991, 15:2557), etc.

In bacterial systems, a number of expression vectors may be advantageously selected depending upon the use intended for the construct being expressed. For example, when a large quantity of such a protein is to be produced, for the generation of pharmaceutical compositions of a construct, vectors that direct the expression of high levels of readily purified fusion protein products may be desirable. Such vectors include, but are not limited to, the E. coli expression vector pUR278 (Ruther et al., EMBO J. 1983, 2:1791), in which the coding sequence may be ligated individually into the vector in frame with the lac Z coding region so that a fusion protein is produced; pIN vectors (Inouye & Inouye, Nucleic Acids Res. 1985, 13:3101-3109; Van Hleeke & Schuster, J. Biol. Chem. 1989, 264:5503-5509); and the like. pGEX vectors may also be used to express foreign polypeptides as fusion proteins with glutathione S-transferase (GST). In general, such fusion proteins are soluble and can easily be purified from lysed cells by adsorption and binding to a matrix of glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example, the polyhedrin gene) of the virus and placed under control of an AcNPV promoter (for example, the polyhedrin promoter).

In mammalian host cells, a number of viral-based and non-viral-based expression systems may be utilized. In cases where an adenovirus is used as an expression vector, the coding sequence of interest may be ligated to an adenovirus transcription/translation control complex, e.g., the late promoter and tripartite leader sequence. This chimeric gene may then be inserted into the adenovirus genome. Insertion in a non-essential region of the viral genome (e.g., region E1 or E3) will result in a recombinant virus that is viable and capable of expressing the construct in infected hosts (see, e.g., Logan and Shenk, Proc. Natl. Acad. Sci. USA 1984, 81:3655-3659). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., Methods in Enzymol. 1987, 153:516-544).

Additionally, a host cell strain may be chosen that modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product may be used. Such mammalian host cells include but are not limited to CHO, VERO, BHK, HeLa, COS, MDCK, 293, 3T3, WI38.

For long-term, high-yield production of recombinant proteins, stable expression is preferred. For example, cell lines that stably express the antibody may be engineered. Rather than using expression vectors that contain viral origins of replication, host cells can be transformed with DNA controlled by appropriate expression control elements (e.g., promoter, enhancer sequences, transcription terminators, polyadenylation sites, etc.) and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci, which, in turn, can be cloned and expanded into cell lines. This method may advantageously be used to engineer cell lines that express the antibody. Such engineered cell lines may be particularly useful in screening and evaluation of compounds that interact directly or indirectly with the antibody.

A number of selection systems may be used, including but not limited to the herpes simplex virus thymidine kinase (Wigler et al., Cell 1977, 11:223), hypoxanthine-guanine phosphoribosyltransferase (Szybalska and Szybalski, Proc. Natl. Acad. Sci. USA 1962, 48:2026), and adenine phosphoribosyltransferase (Lowy et al., Cell 1980, 22:817) genes can be employed in tk-, hgprt-, or aprt-cells, respectively. Also, antimetabolite resistance can be used as the basis of selection for the following genes: dhfr, which confers resistance to methotrexate (Wigler et al, Proc. Natl. Acad. Sci. USA 1980, 77:3567; O'Hare et al., Proc. Natl. Acad. Sci. USA 1981, 78:1527); gpt, which confers resistance to mycophenolic acid (Mulligan and Berg, Proc. Natl. Acad. Sci. USA 1981, 78:2072); neo, which confers resistance to the aminoglycoside G-418 (Colberre-Garapin et al., J. Mol. Biol. 1981, 150:1); and hygro, which confers resistance to hygromycin (Santerre et al., Gene 1984, 30:147).

The expression levels of the construct can be increased by vector amplification (for a review, see Bebbington and Hentschel, The Use of Vectors Based on Gene Ampliflication for the Expression of Cloned Genes in Mammalian Cells in DNA Cloning, Vol. 3., Academic Press, New York, 1987). When a marker in the vector system expressing a construct is amplifiable, increases in the level of inhibitor present in the culture medium of the host cell will increase the number of copies of the marker gene. Since the amplified region is associated with the construct gene, production of the construct will also increase (Crouse et al., Mol. Cell. Biol. 1983, 3:257).

In a specific embodiment in which the construct is an antibody (immunoglobulin), the host cell may be co-transfected with two expression vectors of the invention, the first vector encoding a heavy chain derived polypeptide and the second vector encoding a light chain derived polypeptide. The two vectors may contain identical selectable markers, which enable equal expression of heavy and light chain polypeptides. Alternatively, a single vector may be used that encodes both heavy and light chain polypeptides. In such situations, the light chain should be placed before the heavy chain to avoid an excess of toxic free heavy chain (Proudfoot, Nature 1986, 322:562; Kohler, Proc. Natl. Acad Sci. USA 1980, 77:2197). The coding sequences for the heavy and light chains may comprise cDNA or genomic DNA.

The invention provides a recombinant cell that contains a vector encoding a construct of the invention.

### Viral and Non-Viral Vectors

Preferred vectors, particularly for cellular assays in vitro and in vivo, are viral vectors, such as lentiviruses, retroviruses, herpes viruses, adenoviruses, adeno-associated viruses, vaccinia virus, baculovirus, and other recombinant viruses with desirable cellular tropism; and non-viral vectors. For gene therapy in vivo or ex vivo, a pharmaceutically acceptable vector is preferred, such as a replication incompetent viral vector. Pharmaceutically acceptable vectors containing the nucleic acids of this invention can be further modified for transient or stable expression. As used herein, the term “pharmaceutically acceptable vector” includes, but is not limited to, a vector or delivery vehicle having the ability to selectively target and introduce the nucleic acid into cells.

Thus, a gene encoding a functional or mutant protein or polypeptide domain fragment thereof can be introduced in vivo, ex vivo, or in vitro using a viral vector or through direct introduction of DNA. Expression in targeted tissues can be affected by targeting the transgenic vector to specific cells, such as with a viral vector or a receptor ligand, or by using a tissue-specific promoter, or both. Targeted gene delivery is described in PCT Publication No. WO 95/28494.

Viral vectors commonly used for in vivo or ex vivo targeting and therapy procedures are DNA-based vectors and retroviral vectors. Methods for constructing and using viral vectors are known in the art (see, e.g., Miller and Rosman, BioTechniques 1992, 7:980-990). Preferably, the viral vectors are replication-defective, that is, they are unable to replicate autonomously in the target cell. Preferably, the replication defective virus is a minimal virus, i.e., it retains only the sequences of its genome that are necessary for encapsidating the genome to produce viral particles.

DNA viral vectors include an attenuated or defective DNA virus, such as but not limited to, herpes simplex virus (HSV), papillomavirus, Epstein Barr virus (EBV), adenovirus, adeno-associated virus (AAV), alphavirus (especially Sindbis virus), and the like. Defective viruses that entirely or almost entirely lack viral genes are preferred. Defective virus is not infective after introduction into a cell. Use of defective viral vectors allows for administration to cells in a specific, localized area, without concern that the vector can infect other cells. Thus, a specific tissue can be specifically targeted. Examples of particular vectors include, but are not limited to, a defective herpes virus 1 (HSV1) vector (Kaplitt et al., Molec. Cell. Neurosci. 1991, 2:320-330), defective herpes virus vector lacking a glyco-protein L gene, or other defective herpes virus vectors (PCT Publication Nos. WO 94/21807 and WO 92/05263); an attenuated adenovirus vector, such as the vector described by Stratford-Perricaudet et al. (J. Clin. Invest. 1992, 90:626-630; see also La Salle et al., Science 1993, 259:988-990); a defective adeno-associated virus vector (Samulski et al., J. Virol., 1987, 61:3096-3101; Samulski et al., J. Virol. 1989, 63:3822-3828; Lebkowski et al., Mol. Cell. Biol. 1988, 8:3988-3996); and Alphavirus vectors, including Sindbis virus and Semlild Forest virus-based vectors (U.S. Pat. No. 5,091,309; PCT Publication No. WO 98/44132; Schlesinger and Dubensky, Curr. Opin. Biotechnol. 1999, 5:434-9; Zaks et al., Nat. Med. 1999, 7:823-7).

Various companies produce viral vectors commercially, including, but not limited to, Avigen, Inc. (Alameda, Calif.; AAV vectors), Cell Genesys (Foster City, Calif.; retroviral, adenoviral, AAV, and lentiviral vectors), Clontech (retroviral and baculoviral vectors), Genovo, Inc. (Sharon Hill, Pa.; adenoviral and AAV vectors), Genvec (France; adenoviral vectors), IntroGene (Leiden, Netherlands; adenoviral vectors), Molecular Medicine (retroviral, adenoviral, AAV, and herpes viral vectors), Norgen (adenoviral vectors), Oxford BioMedica (Oxford, United Kingdom; lentiviral vectors), and Transgene (Strasbourg, France; adenoviral, vaccinia, retroviral, and lentiviral vectors).

Adenovirus vectors. Adenoviruses are eukaryotic DNA viruses that can be modified to efficiently deliver a nucleic acid of the invention to a variety of cell types. Various serotypes of adenovirus exist. Of these serotypes, preference is given, within the scope of the present invention, to using type 2 or type 5 human adenoviruses (Ad 2 or Ad 5; see PCT Publication No. WO 95/27071) or adenoviruses of animal origin (see PCT Publication No. WO 94/26914). Those adenoviruses of animal origin that can be used within the scope of the present invention include adenoviruses of canine, bovine, murine (example: Mav1, Beard et al., Virology 1991, 180:257-65), ovine, porcine, avian, and simian (example: SAV) origin. Preferably, the adenovirus of animal origin is a canine adenovirus, more preferably a CAV2 adenovirus (e.g., Manhattan or A26/61 strain, ATCC VR-800, for example). Various replication defective adenovirus and minimum adenovirus vectors have been described (PCT Publication Nos. WO 94/26914, WO 95/02697, WO 94/28938, WO 94/28152, WO 94/12649, WO 95/00655, WO 95/02697, WO 95/11984, WO 96/22378; Virology 1988, 163:614-7). The replication defective recombinant adenoviruses according to the invention can be prepared by any technique known to the person skilled in the art (Levrero et al., Gene, 1991, 101:195; European Publication No. EP 185 573; Graham, EMBO J., 1984, 3:2917; Graham et al., J. Gen. Virol., 1977, 36:59). Recombinant adenoviruses are recovered and purified using standard molecular biological techniques that are well known to one of ordinary skill in the art.

Adeno-associated viruses. The adeno-associated viruses (AAV) are DNA viruses of relatively small size that can integrate, in a stable and site-specific manner, into the genome of the cells that they infect. They are able to infect a wide spectrum of cells without inducing any effects on cellular growth, morphology or differentiation, and they do not appear to be involved in human pathologies. The AAV genome has been cloned, sequenced and characterized. The use of vectors derived from the AAVs for transferring genes in vitro and in vivo has been described (see, PCT Publication Nos. WO 91/18088 and WO 93/09239; U.S. Pat. Nos. 4,797,368 and 5,139,941; European Publication No. EP 488 528; Hermonat and Muzyczka, Proc. Natl. Acad. Sci. USA 1984, 81:6466-70; Lebkowski et al, Mol. Cell. Biol. 1988, 8:3988-96). The replication defective recombinant AAVs according to the invention can be prepared by cotransfecting a plasmid containing the nucleic acid sequence of interest flanked by two AAV inverted terminal repeat (ITR) regions, and a plasmid carrying the AAV encapsidation genes (rep and cap genes), into a cell line that is infected with a human helper virus (for example an adenovirus). The AAV recombinants that are produced are then purified by standard techniques.

Retrovirus vectors. In another embodiment the gene can be introduced in a retroviral vector (see, e.g., U.S. Pat. No. 5,399,346; Mann et al., Cell 1983, 33:153; U.S. Pat. Nos. 4,650,764 and 4,980,289; Markowitz et al., J. Virol. 1988, 62:1120; U.S. Pat. No. 5,124,263; European Publication Nos. EP 453 242 and EP178 220; Bernstein et al., Genet. Eng.1985, 7:235; McCormick, BioTechnology 1985, 3:689; PCT Publication No. WO 95/07358; and Kuo et al., Blood 1993, 82:845; Correll et al., Proc. Natl. Acad. Sci. USA 1989, 86:8912-6; Bordignon, Proc. Natl. Acad. Sci. USA 1989, 86:6748-52; Culver, Proc. Natl. Acad. Sci. USA 1991, 88:3155; Rill, Blood 1991, 79:2694-2700; Miller et al., BioTechniques 1989, 7:980-90 [LnL6 replication-incompetent retroyiral vector]). The retroviruses are integrating viruses that infect dividing cells. The retrovirus genome includes two LTRs, an encapsidation sequence and three coding regions (gag, pol and env). In recombinant retroviral vectors, the gag, pol and env genes are generally deleted, in whole or in part, and replaced with a heterologous nucleic acid sequence of interest. These vectors can be constructed from different types of retrovirus, such as, HIV, MoMuLV (“murine Moloney leukemia virus”) MSV (“murine Moloney sarcoma virus”), HaSV (“Harvey sarcoma virus”); SNV (“spleen necrosis virus”); RSV (“Rous sarcoma virus”) and Friend virus. Suitable packaging cell lines have been described in the prior art, in particular the cell line PA317 (U.S. Pat. No. 4,861,719); the PsiCRIP cell line (PCT Publication No. WO 90/02806) and the GP+envAm-12 cell line (PCT Publication No. WO 89/07150). In addition, the recombinant retroviral vectors can contain modifications within the LTRs for suppressing transcriptional activity as well as extensive encapsidation sequences that may include a part of the gag gene (Bender et al., J. Virol. 1987, 61:1639). Recombinant retroviral vectors are purified by standard techniques known to those having ordinary skill in the art.

Retroviral vectors can be constructed to function as infectious particles or to undergo a single round of transfection. In the former case, the virus is modified to retain all of its genes except for those responsible for oncogenic transformation properties, and to express the heterologous gene. Non-infectious viral vectors are manipulated to destroy the viral packaging signal, but retain the structural genes required to package the co-introduced virus engineered to contain the heterologous gene and the packaging signals. Thus, the viral particles that are produced are not capable of producing additional virus.

Retrovirus vectors can also be introduced by DNA viruses, which permit one cycle of retroviral replication and amplifies transfection efficiency (see PCT Publication Nos. WO 95/22617, WO 95/26411, WO 96/39036 and WO 97/19182).

Lentivirus vectors. In another embodiment, lentiviral vectors can be used as agents for the direct delivery and sustained expression of a transgene in several tissue types, including brain, retina, muscle, liver and blood. The vectors can efficiently transduce dividing and nondividing cells in these tissues, and maintain long-term expression of the gene of interest. For a review, see, Naldini, Curr. Opin. Biotechnol. 1998, 9:457-63; see also Zufferey, et al., J. Virol. 1998, 72:9873-80). Lentiviral packaging cell lines are available and known generally in the art. They facilitate the production of high-titer lentivirus vectors for gene therapy. An example is a tetracycline-inducible VSV-G pseudotyped lentivirus packaging cell line that can generate virus particles at titers greater than 10⁶ IU/ml for at least 3 to 4 days (Kafri, et al., J. Virol. 1999, 73: 576-584). The vector produced by the inducible cell line can be concentrated as needed for efficiently transducing non-dividing cells in vitro and in vivo.

Non-viral vectors. In another embodiment, the vector can be introduced in vivo by lipofection, as naked DNA, or with other transfection facilitating agents (peptides, polymers, etc.). Synthetic cationic lipids can be used to prepare liposomes for in vivo transfection of a gene encoding a marker (Felgner, et. al., Proc. Natl. Acad. Sci. USA 1987, 84:7413-7417; Felgner and Ringold, Science 1989, 337:387-388; see Mackey, et al., Proc. Natl. Acad. Sci. USA 1988, 85:8027-8031; Ulmer et al., Science 1993,259:1745-1748). Useful lipid compounds and compositions for transfer of nucleic acids are described in PCT Patent Publication Nos. WO 95/18863 and WO 96/17823, and in U.S. Pat. No. 5,459,127. Lipids may be chemically coupled to other molecules for the purpose of targeting (see Mackey, et. al., supra). Targeted peptides, e.g., hormones or neurotransmitters, and proteins such as antibodies, or non-peptide molecules could be coupled to liposomes chemically.

Other molecules are also useful for facilitating transfection of a nucleic acid in vivo, such as a cationic oligopeptide (e.g., PCT Patent Publication No. WO 95/21931), peptides derived from DNA binding proteins (e.g., PCT Patent Publication No. WO 96/25508), or a cationic polymer (e.g., PCT Patent Publication No. WO 95/21931).

It is also possible to introduce the vector in vivo as a naked DNA plasmid. Naked DNA vectors for gene therapy can be introduced into the desired host cells by methods known in the art, e.g., electroporation, microinjection, cell fusion, DEAE dextran, calcium phosphate precipitation, use of a gene gun, or use of a DNA vector transporter (see, e.g. Wu et al., J. Biol. Chem. 1992, 267:963-967; Wu and Wu, J. Biol. Chem. 1988, 263:14621-14624; Canadian Patent Application No. 2,012,31 1; Williams et al., Proc. Natl. Acad. Sci. USA 1991, 88:2726-2730). Receptor-mediated DNA delivery approaches can also be used (Curiel et al., Hum. Gene Ther. 1992, 3:147-154; Wu and Wu, J. Biol. Chem. 1987, 262:4429-4432). U.S. Pat. Nos. 5,580,859 and 5,589,466 disclose delivery of exogenous DNA sequences, free of transfection facilitating agents, in a mammal. Recently, a relatively low voltage, high efficiency in vivo DNA transfer technique, termed electrotransfer, has been described (Mir et al., C.P. Acad. Sci. 1.988, 321:893; PCT Publication Nos. WO 99/01157, WO 99/01158, and WO 99/01175).

### Therapeutic Use of Constructs

The invention also provides methods for treating adenocarcinomas by administration of a therapeutic of the invention. Such therapeutics include the constructs of the invention and nucleic acids encoding the constructs of the invention.

Generally, administration of products of a species origin or species reactivity that is the same species as that of the subject is preferred. Thus, in administration to humans, the therapeutic methods of the invention preferably use a construct that is derived from a human immunoglobulin superfamily protein but may be an immunoglobulin superfamily protein from a heterologous species such as, for example, a mouse, which may or may not be humanized; in other embodiments, the methods of the invention use a modified antibody that is derived from a chimeric or humanized antibody.

Specifically, therapeutic compositions containing the constructs of the invention that specifically bind a particular molecule can be used in the treatment or prevention of diseases or disorders associated with the expression of the particular ligand polypeptide.

The subjects to which the present invention is applicable may be any mammalian or vertebrate species that include, but are not limited to, cows, horses, sheep, pigs, fowl (e.g., chickens), goats, cats, dogs, hamsters, mice, rats, monkeys, rabbits, chimpanzees, and humans. In a preferred embodiment, the subject is a human.

### Treatment and Prevention of Cancers

The invention provides methods of treating or preventing cancers. The method includes administering to a subject in need of such treatment or prevention a therapeutic of the invention, i.e., a construct, that comprises a variable domain with a CDR containing the antigenic amino acid sequence of a ligand having immunogenic properties relevant to human lung cancer, or a nucleic acid vector encoding such a construct.

Cancers, including, but not limited to, neoplasms, tumors, metastases, or any disease or disorder characterized by uncontrolled cell growth, in which the tumor cells express a ligand as described herein having immunogenic properties relevant to human lung cancer, can be treated or prevented by administration of a construct of the invention. Whether a particular therapeutic is effective to treat or prevent a certain type of cancer can be determined by any method known in the art, for example but not limited to, the methods described in infra.

In other embodiments of the invention, the subject being treated with the construct may, optionally, be treated with other cancer treatments such as surgery, radiation therapy, or chemotherapy. In particular, the therapeutic of the invention used to treat or prevent cancer may be administered in conjunction with one or a combination of chemotherapeutic agents including, but not limited to, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposides, campathecins, bleomycin, doxormbicin, idarubicin, daunorubicin, dactinomycin, plicamycin, mitoxantrone, asparaginase, vinblastine, vincristine, vinorelbine, paclitaxel, docetaxel, etc.

### Gene Vaccines

In a specific embodiment, vectors comprising a sequence encoding a construct of the invention are administered to treat or prevent a disease or disorder associated with the expression or function of a molecule to which the construct specifically binds.

Any of the methods for gene therapy available in the art can be used according to the present invention. Exemplary methods are described below.

For general reviews of the methods of gene therapy, see, Goldspiel et al., Clinical Pharmacy 1993, 12:488-505; Wu and Wu, Biotherapy 1991, 3:87-95; Tolstoshev, Ann. Rev. Pharmacol. Toxicol. 1993, 32:573-596; Mulligan, Science 1993, 260:926-932; and Morgan and Anderson, Ann. Rev. Biochem. 1993, 62:191-217; and May, TIBTECH 1993, 11:155-215. Methods commonly known in the art of recombinant DNA technology that can be used are described in Ausubel et al., (eds.), 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al., (eds.), 1994, Current Protocols in Human Genetics, John Wiley & Sons, NY. Vectors suitable for gene therapy are described above.

In one aspect, the therapeutic vector comprises a nucleic acid that expresses the construct in a suitable host. In particular, such a vector has a promoter operationally linked to the coding sequence for the construct. The promoter can be inducible or constitutive and, optionally, tissue-specific. In another embodiment, a nucleic acid molecule is used in which the antibody coding sequences and any other desired sequences are flanked by regions that promote homologous recombination at a desired site in the genome, thus providing for expression of the construct from a nucleic acid molecule that has integrated into the genome (Koller and Smithies, Proc. Natl. Acad. Sci. USA 1989, 86:8932-8935; Zijlstra et al., Nature 1989, 342:435-438).

Delivery of the vector into a patient may be either direct, in which case the patient is directly exposed to the vector or a delivery complex, or indirect, in which case, cells are first transformed with the vector in vitro then transplanted into the patient. These two approaches are known, respectively, as in vivo and ex vivo gene therapy.

In a specific embodiment, the vector is directly administered in vivo, where it enters the cells of the organism and mediates expression of the constructs. This can be accomplished by any of numerous methods known in the art, e.g., by constructing it as part of an appropriate expression vector and administering it so that it becomes intracellular, e.g., by infection using a defective or attenuated retroviral or other viral vector (see, U.S. Pat. No. 4,980,286), or by direct injection of naked DNA, or by use of microparticle bombardment (e.g. a gene gun; Biolistic, Dupont); or coating with lipids or cell-surface receptors or transfecting agents, encapsulation in biopolymers (e.g., poly-β1-4-N-acetylglucosamine polysaccharide; see, U.S. Pat. No. 5,635,493), encapsulation in liposomes, microparticles, or microcapsules; by administering it in linkage to a peptide or other ligand known to enter the nucleus; or by administering it in linkage to a ligand subject to receptor-mediated endocytosis (see, e.g., Wu and Wu, J. Biol. Chem. 1987, 62:44294432), etc. In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publication Nos. WO 92/06180, WO 92/22635, WO 92/20316 and WO 93/14188). These methods are in addition to those discussed above in conjunction with “Viral and Non-viral Vectors”.

Alternatively, single chain antibody-like constructs can also be administered, for example, by expressing nucleotide sequences encoding single-chain antibodies within the target cell population by utilizing, for example, techniques such as those described in Marasco et al. (Proc. Natl. Acad Sci. USA 1993, 90:7889-7893).

The form and amount of therapeutic nucleic acid envisioned for use depends on the type of disease and the severity of the desired effect, patient state, etc., and can be determined by one skilled in the art.

### Vaccine Formulations and Administration

The invention also provides vaccine formulations containing therapeutic constructs of the invention, which vaccine formulations are suitable for administration to elicit a protective immune (humoral and/or cell mediated) response against cancer cells bearing a p53BP2 ligand as described herein, e.g., for the treatment and prevention of diseases.

Suitable preparations of such vaccines include injectables, either as liquid solutions or suspensions; solid forms suitable for solution in, suspension in, liquid prior to injection, may also be prepared. The preparation may also be emulsified, or the constructs antibodies encapsulated in liposomes. The active immunogenic ingredients are often mixed with excipients which are pharmaceutically acceptable and compatible with the active ingredient. Suitable excipients are, for example, water, saline, buffered saline, dextrose, glycerol, ethanol, sterile isotonic aqueous buffer or the like and combinations thereof. In addition, if desired, the vaccine preparation may also include minor amounts of auxiliary substances such as wetting or emulsifying agents, pH buffering agents, and/or adjuvants that enhance the effectiveness of the vaccine. The construct when prepared as a vaccine can be introduced in microspheres or microcapsules, e.g., prepared from PGLA (see, U.S. Pat. Nos. 5,814,344, 5,100,669, and 4,849,222; PCT Publication Nos. WO 95/11010 and WO 93/07861).

The effectiveness of an adjuvant may be determined by measuring the induction of anti-idiotype antibodies directed against the injected construct formulated with the particular adjuvant.

The composition can be a liquid solution, suspension, emulsion, tablet, pill, capsule, sustained release formulation, or powder. For oral administration, the therapeutics can take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well known in the art. Liquid preparations for oral administration can take the form of, for example, solutions, syrups, emulsions or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

Generally, the ingredients are supplied either separately or mixed together in unit dosage form, for example, as a dry lyophilized powder or water-free concentrate in a sealed container such as a vial or sachette indicating the quantity of active agent. Where the composition is administered by injection, an ampoule of sterile diluent can be provided so that the ingredients may be mixed prior to administration.

In a specific embodiment, the lyophilized construct of the invention is provided in a first container; a second container comprises diluent consisting of an aqueous solution of 50% glycerin, 0.25% phenol, and an antiseptic (e.g., 0.005% brilliant green).

The invention also provides a pharmaceutical pack or kit comprising one or more containers filled with one or more of the ingredients of the vaccine formulations of the invention. Associated with such container(s) can be a notice in the form prescribed by a governmental agency regulating the manufacture, use or sale of pharmaceuticals or biological products, which notice reflects approval by the agency of manufacture, use or sale for human administration.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration. Composition comprising a compound of the invention formulated in a compatible pharmaceutical carrier may also be prepared, placed in an appropriate container, and labeled for treatment of an indicated condition.

Many methods may be used to introduce the vaccine formulations of the invention; these include but are not limited to oral, intracerebral, intradermal, intramuscular, intraperitoneal, intravenous, subcutaneous, intranasal routes, and via scarification (scratching through the top layers of skin, e.g., using a bifurcated needle) or any other standard routes of immunization.

### Effective Dose

The constructs and vectors described herein can be administered to a patient at therapeutically effective doses to treat p53BP2 ligand-bearing cancers. A therapeutically effective dose refers to that amount of a therapeutic sufficient to result in a healthful benefit in the treated subject.

The precise dose of the constructs to be employed in the formulation depends on the route of administration, and the nature of the patient's cancer, and should be decided according to the judgment of the practitioner and each patient's circumstances according to standard clinical techniques. An effective dose is an amount effective to result in development of an effective anti-tumor immune response in vivo. The ability of a therapeutic composition of the invention to produce this effect can be detected in vitro, e.g. using a binding assay with labeled construct as exemplified infra. Such an assay can be formatted in a solid phase format or in a cell-based assay format. Effective doses may be extrapolated from dose-response curves derived from animal model test systems, including transgenic animal models.

Toxicity and therapeutic efficacy of compounds can be determined by standard pharmaceutical procedures in cell cultures or experimental animals, e.g. for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Therapeutics that exhibit large therapeutic indices are preferred. While therapeutics that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage lies preferably within a range of circulating concentrations that include the ED₅₀ with little or no toxicity. The dosage can vary within this range depending upon the dosage form employed and the route of administration utilized. For any construct used in the method of the invention, the therapeutically effective dose can be estimated initially from cell culture assays. A dose can be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma can be measured, for example, by high performance liquid chromatography.

### EXAMPLES

The following Examples illustrate the invention without limiting it.

### Example 1

### Construction of Variable Region Gene Containing the CDR Sequences from p53BP2

Heavy and light chain variable region genes are constructed containing consensus framework sequences and CDR sequences from the novel ligand p53BP2. The engineered genes are made by assembling overlapping oligonucleotides that are from 65 to 72 nucleotides in length using standard conditions. A second set of heavy and light chain variable region genes are also constructed in which specific cysteine residues, known to form intra-chain disulfide bonds, are changed to alanine residues. Cysteine residues at positions 22 and 96 of the heavy chain and 23 and 88 of the light chain are changed to alanine residues. The assembled variable region genes are joined to appropriate constant region genes and then inserted into an expression vector.

To construct the variable region genes encoding the ligand sequences and lacking the intra-chain disulfide bonds, the following steps are performed. Purified, single stranded oligodeoxynucleotides are annealed together to create cohesive, double stranded DNA fragments. The specific sequences of the oligonucleotides used to construct heavy and light chain variable region genes containing the sequences from the ligand p53BP2. The double stranded DNA fragments have cohesive, single stranded ends of six to nine bases that are required for joining the individual fragments in the following steps. In the next step, two annealed, double stranded DNA fragments are ligated together using standard conditions and this process is continued until the assembly of the full-length variable region gene is completed. The mixture of ligated fragments is then separated on an agarose gel and the full-length fragment is isolated and purified using a QIAEX II gel extraction kit according to the manufacturer's instructions.

To change the alanine residues present in the variable region genes back to cysteine residues, oligodeoxynucleotides are prepared and used with a QuikChange site-directed mutagenesis kit from Stratagene according to the manufacturer's instructions. The amino acid changes are confirmed by DNA sequencing using an ABI 310 Genetic Analyzer.

The assembled, modified variable region genes containing the ligand sequences are then linked to the appropriate constant region clones. For assembly of the heavy chain of the antibody, a unique XhoI restriction enzyme site is engineered into both the 3′end of the variable region and the 5′ end of the IgG₁ heavy chain constant region. At the 5′end of the variable region, an EcoRI restriction site and a Kozak sequence are added using polymerase chain reactions (PCR). The modified heavy chain variable region is then joined to the heavy chain constant region by inserting the EcoRI/XhoI cut variable region fragment into a vector containing the EcoRI/XhoI cut heavy chain constant region

For assembly of the light chain of the antibody, a unique BglII restriction enzyme site is engineered into the 3′end of the light chain variable region and a BclI restriction enzyme site is added to the 5′end of the light chain constant region (k chain). Similar to the heavy chain variable region, an EcoRI restriction site and Kozak sequence are added to the 5′end of the light chain variable region using PCR. When BglII and BclI cut their respective cleavage sites, both enzymes leave overhangs with the same DNA sequence, which allows them to be ligated. Consequently, the modified light chain variable region clone is digested with EcoRI/BglII and the resulting fragment inserted into a vector containing the EcoRI/BclI cut light chain constant region.

In a final step, the heavy chain expression vector, containing the heavy chain variable region, and the light chain expression vector, containing the light chain variable region, are assembled into a single “double gene” expression vector. To assemble the “double gene” vector, the heavy chain expression vector is cleaved with BamHI and NotI. The resulting fragment contains the complete heavy chain expression cassette including the CMV promoter, the assembled heavy chain and a transcriptional terminator. The light chain expression vector is also cleaved with BamHI and NotI and after purifying the vector from a small fragment, the heavy chain expression cassette is inserted into the light chain vector.

### Example 2

### Construction of Epitope String Containing Amino Acid Sequences From p53

An epitope string containing epitopes of p53BP2 is constructed with the standard cloning steps and conditions.

The following p53BP2 epitopes are incorporated into epitope string constructs:

APC (Antigen Presenting Cell) Targeting Sequences:

(1) The Mannose Receptor on Antigen Presenting Cells:
peptide mimetics of mannose: Y P Y
Oldenburg, K R, et al (1992) Peptide ligands for a sugar-binding protein isolated from a random peptide library, Proc. Natl. Acad. Sci. USA 89 5393-5397

(2) The Fcγ Receptor on Antigen Presenting Cells:
peptides from the hinge region of antibodies: C P A P E L L G G P S V
Radaev, S and Sun, P D (2001) Recognition of IgG by Fcγ Receptor J. Biol. Chem. 276 16478-16483

(3) Other Antigen Presenting Cell Selective Membrane Proteins:
DEC-205, Mahnke, K, et al (2000); the dendritic cell receptor for endocytosis;
DEC-205, can recycle and enhance antigen presentation via major histocompatibility complex class II-positive lysosomal compartments, J. Cell Biol. 151, 673-683 β₂ microglobulin.
   p53BP2 Epitope Sequences:

CTL Epitopes:
HLA-A1 V L D V Y L E E Y
   G P N I Q K L L Y
   Y T Q C S Q F L Y
HLA-A2 SEQ ID NUMBERS: 3, 5, 7, 9, 11, 13, 15, 17, 19 and 21
HLA-A3 Q V A N K N V A K
   K L K K V R A L K
   T V V P S M G T K

T-Helper Epitopes:
DRB1 0101 G P H F S S V Y G K P V I A A
   I V K F L V Q F G V N V N A A
   A D L F P S Q G S A S V P Q S
A0201 L L S P S I P S V
   F L V E S G A A V
   F L V Q F G V N V
      Non-Specific Epitope Sequences

T-helper epitopes:
influenza PR8 A virus hemaglutinin peptide 110-120:
   S F E R F E I F P K E
Brumeanu, T D, Dehazya, P. Wolf, I and Bona, C A (1995)
Enzymatically mediated, glycosidic conjugation of immunoglobulins with vital epitopes J. Immunological Methods 183, 185-197
HIV-1 T-Helper Epitope:
K Q I I N M W Q E V G K A M Y A P P I S G Q I R
U.S. Pat. No. 5,932,218

The p53 epitope sequences are then linked to the appropriate targeting sequences, such as

[LeuXaaXaaAspXaaXaaPro][XaaLysXaaLysPhe]-[p53 epitope]-[LeuXaaXaaAspXaaXaaPro][XaaLysXaaLysPhe]-[p53 epitope]-[p80], wherein Xaa represents an amino acid.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

All patents, applications, publications, test methods, literature, and other materials cited herein are hereby incorporated by reference.

What is claimed is:

1. A variant of an immunoglobulin variable domain, said immunoglobulin variable domain comprising (A) at least one CDR region and (B) framework regions flanking said CDR, said variant comprising:
(a) said CDR region having added or substituted therein at least one binding sequence and
(b) said flanking framework regions,
wherein said binding sequence is heterologous to said CDR and is an antigenic sequence from p53 binding protein having immunogenic properties relevant to lung human cancer.

2. The variant as define in claim 1, wherein the variable domain lacks an intrachain disulfide bond.

3. A variant as defined in claim 1, wherein (i) one or more amino acid residues in one or more of said flanking framework regions has been substituted or deleted, (ii) one or more amino acid residues has been added in one or more of said flanking framework regions, or (iii) a combination of (i) and (ii).

4. A variant as defined in claim 1, wherein (i) one or more amino acid residues in one or more framework regions other than said framework regions flanking said CDR has been substituted or deleted, (ii) one or more amino acid residues has been added in one or more framework regions other than said framework regions flanking said CDR, or (iii) a combination of (i) and (ii).

5. A variant as defined in claim 1, wherein (i) one or more amino acid residues in one or more of said flanking framework regions has been substituted or deleted, (ii) one or more amino acid residues has been added in one or more of said flanking framework regions, or (iii) a combination of (i) and (ii); and wherein (iv) one or more amino acid residues in one or more framework regions other than said framework regions flanking said CDR has been substituted or deleted, (v) one or more amino acid residues has been added in one or more framework regions other than said framework regions flanking said CDR, or (vi) a combination of (iv) and (v).

6. A variant of an immunoglobulin variable domain, said immunoglobulin variable domain comprising (A) at least one CDR region and (B) framework regions flanking said CDR, said variant comprising:
(a) said CDR region having added or substituted therein at least one amino acid sequence which is heterologous to said CDR and
(b) said flanking framework regions,
wherein said heterologous sequence is an antigenic sequence from a p53 binding protein having immunogenic properties relevant to lung human cancer.

7. A variant as defined in claim 6, wherein the variable domain lacks an intrachain disulfide bond.

8. A variant as defined in claim 6, wherein (i) one or more amino acid residues in one or more of said flanking framework regions has been substituted or deleted, (ii) one or more amino acid residues has been added in on or more of said flanking framework regions, or (iii) a combination of (i) and (ii).

9. A variant as defined in claim 6, wherein (i) one or more amino acid residues in one or more framework regions other than said framework regions flanking said CDR has been substituted or deleted, (ii) one or more amino acid residues has been added in one or more framework regions other than said framework regions flanking said CDR, or (iii) a combination of (i) and (ii).

10. A variant as defined in claim 6, wherein (i) one or more amino acid residues in one or more of said flanking framework regions has been substituted or deleted, (ii) one or more amino acid residues has been added in one or more of said flanking framework regions, (iii) a combination of (i) and (ii); and wherein (iv) one or more amino acid residues in one or more framework regions other than said framework regions flanking said CDR has been substituted or deleted, (v) one or more amino acid residues has been added in one or more framework regions other than said framework regions flanking said CDR, or (vi) a combination of (iv) and (v).

11. A variant as defined in claim 6, wherein said CDR is more than one CDR.

12. A variant as defined in claim 6, wherein said heterologous sequence is a CDR of a heavy chain variable region.

13. A variant as defined in claim 6, wherein said heterologous sequence is a CDR of a light chain variable region.

14. A variant as defined in claim 6, wherein said antigenic sequence is selected from the group consisting of SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, amino acids 669 to 677 of SEQ ID NO: 2, and SEQ ID NO: 21.

15. A variant as defined in claim 6, which is an antibody.

16. A molecule comprising a variant as defined in claim 6.

17. A molecule comprising a variant as defined in claim 7.

18. A molecule comprising a variant as defined in claim 8.

19. A molecule comprising a variant as defined in claim 9.

20. A molecule comprising a variant as defined in claim 10.

21. A molecule comprising a variant as defined in claim 14.

22. A molecule as defined in claim 16, further comprising one or more constant domains from an immunoglobulin.

23. A molecule as defined in claim 16, further comprising a second variable domain linked to said variant.

24. A molecule as defined in claim 16, further comprising a second variable domain linked to said variant, and one or more constant domains from an immunoglobulin.

25. A molecule as defined in claim 16, wherein said CDR region is CDR 1.

26. A molecule as defined in claim 16, wherein said CDR region is CDR 2.

27. A molecule as defined in claim 16, wherein said CDR region is CDR 3.

28. A molecule as defined in claim 16, which is an antibody.

29. A molecule as defined in claim 16, which is derived from a human antibody.

30. A molecule as defined in claim 16, which is derived from a chimeric or a humanized antibody.

31. An immunoglobulin comprising a heavy chain and a light chain, wherein said heavy chain comprises a variant as defined in claim 6 and three constant domains from an immunoglobulin heavy chain, and said light chain comprises a second variable domain associated with said variant and a constant domain from an immunoglobulin light chain.

32. An immunoglobulin comprising a heavy chain and a light chain, wherein said light chain comprises a variant as defined in claim 6 and a constant domain from an immunoglobulin light chain, and said heavy chain comprises a second variable domain associated with said variant and three constant domains from an immunoglobulin heavy chain.

33. An isolated nucleic acid encoding a variant as defined in claim 1.

34. An isolated nucleic acid encoding a variant as defined in claim 6.

35. An isolated nucleic acid encoding a molecule as defined in claim 16.

36. An isolated nucleic acid encoding an immunoglobulin as defined in claim 29.

37. An isolated nucleic acid encoding an immunoglobulin as defined in claim 30.

38. A cell containing nucleic acid as defined in claim 31.

39. A cell containing nucleic acid as defined in claim 32.

40. A cell containing nucleic acid as defined in claim 33.

41. A cell containing nucleic acid as defined in claim 34.

42. A cell containing nucleic acid as defined in claim 35.

43. A recombinant non-human host containing nucleic acid as defined in claim 31.

44. A recombinant non-human host containing nucleic acid as defined in claim 32.

45. A recombinant non-human host containing nucleic acid as defined in claim 33.

46. A recombinant non-human host containing nucleic acid as defined in claim 34.

47. A recombinant non-human host containing nucleic acid as defined in claim 35.

48. A vaccine composition comprising a therapeutically or prophylactically effective amount of a variant as defined in claim 1, and an adjuvant.

49. A vaccine composition comprising a therapeutically or prophylactically effective amount of a variant as defined in claim 6, and an adjuvant.

50. A vaccine composition comprising a therapeutically or prophylactically effective amount of a variant as defined in claim 16, and an adjuvant.

51. A vaccine composition comprising a therapeutically or prophylactically effective amount of an immunoglobulin as defined in claim 29, and an adjuvant.

52. A vaccine composition comprising a therapeutically or prophylactically effective amount of an immunoglobulin as defined in claim 30, and an adjuvant.

53. A method of treating or preventing a lung cancer tumor in a subject in need of such treatment or prevention, said method comprising administering to said subject a disease treating or preventing effective amount of a variant as defined in claim 1.

54. A method of treating or preventing a lung cancer tumor in a subject in need of such treatment or prevention, said method comprising administering to said subject a disease treating or preventing effective amount of a variant as defined in claim 6.

55. A method of treating or preventing a lung cancer tumor in a subject in need of such treatment or prevention, said method comprising administering to said subject a disease treating or preventing effective amount of a molecule as defined in claim 16.

56. A method of treating or preventing a lung cancer tumor in a subject in need of such treatment or prevention, said method comprising administering to said subject a disease treating or preventing effective amount of an immunoglobulin as defined in claim 29.

57. A method of treating or preventing an adenocarcinoma tumor in a subject in need of such treatment or prevention, said method comprising administering to said subject a disease treating or preventing effective amount of an immunoglobulin as defined in claim 30.

58. A method of treating or preventing a lung cancer tumor in a subject in need of such treatment or prevention, said method comprising administering to said subject a disease treating or preventing effective amount of a nucleic acid as defined in claim 31.

59. A method of treating or preventing a lung cancer tumor in a subject in need of such treatment or prevention, said method comprising administering to said subject a disease treating or preventing effective amount of a vaccine composition as defined in claim 46.

60. A method of treating or preventing a lung cancer tumor in a subject in need of such treatment or prevention, said method comprising administering to said subject a disease treating or preventing effective amount of a vaccine as defined in claim 48.

61. A variant of an immunoglobulin variable domain, said immunoglobulin variable domain comprising at least one CDR region, said variant comprising said CDR region having added or substituted therein at least one antigenic sequence from a p53 binding protein having immunogenic properties relevant to human lung cancer, said at least one sequence being selected from the group consisting of (a) a binding sequence heterologous to said CDR; (b) a CTL-epitope sequence; (c) a T-helper cell sequence; (d) a B-helper cell sequence; and (e) combinations thereof, wherein said at least one sequence is heterologous to said CDR and the variable domain lacks an intrachain disulfide bond.

62. A variant as claimed in claim 59 wherein said variable region comprises (a) a CDR1 region having said CTL epitope sequence substituted or added therein; (b) a CDR2 region having said T-helper cell substituted or added therein; and (c) a CDR3 region having said binding sequence of B-helper cell sequence substituted or added therein.

63. A variant as claimed in claim 59 wherein said CTL sequence is selected from the group consisting of HLA-A1, HLA-A2, and HLA-A3.

64. A variant as claimed in claim 59 wherein said T-helper cell sequence is DRB1 0101, and A 0201.

65. A variant as claimed in claim 59 wherein said binding sequence is selected from the group consisting of SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, amino acids 669 to 677 of SEQ ID NO: 2, and SEQ ID NO: 21.

66. A variant as claimed in claim 59 which is an antibody.

67. A molecule comprising a variant as claimed in claim 59.

68. A molecule as claimed in claim 67 further comprising one or more constant domains from an immunoglobulin.

69. A molecule as claimed in claim 67 further comprising a second variable domain linked to said variant.

70. A molecule as claimed in claim 67 further comprising a second variable domain linked to said variant and one or more constant domains from an immunoglobulin.

71. A molecule as claimed in claim 67 which is an antibody.

72. A molecule as claimed in claim 67 which is derived from a human antibody.

73. A molecule as claimed in claim 67 which is derived from a chimeric or humanized antibody.

74. An immunoglobulin comprising a heavy chain and a light chain, wherein said heavy chain comprises a variant as claimed in claim 59 and three constant domains from an immunoglobulin heavy chain, and said light chain comprises a second variable domain associated with said variant and a constant domain from an immunoglobulin light chain.

75. An immunoglobulin comprising a heavy chain and a light chain, wherein said light chain comprises a variant as claimed in claim 59 and a constant domain from an immunoglobulin light chain, and said heavy chain comprises a second variable domain associated with said variant and three constant domains from an immunoglobulin heavy chain.

76. An isolated nucleic acid encoding a variant as claimed in claim 59.

77. An isolated nucleic acid encoding a molecule as claimed in claim 67.

78. An isolated nucleic acid encoding an immunoglobulin as claimed in claim 73.

79. An isolated nucleic acid encoding an immunoglobulin as claimed in claim 74.

80. A cell containing nucleic acid as claimed in claim 75.

81. A cell containing nucleic acid as claimed in claim 76.

82. A cell containing nucleic acid as claimed in claim 77.

83. A cell containing nucleic acid as claimed in claim 78.

84. A recombinant non-human host containing nucleic acid as claimed in claim 75.

85. A recombinant non-human host containing nucleic acid as claimed in claim 76.

86. A recombinant non-human host containing nucleic acid as claimed in claim 77.

87. A recombinant non-human host containing nucleic acid as claimed in claim 78.

88. A vaccine composition comprising a therapeutically or prophylactically effective amount of a variant as claimed in claim 59 and an adjuvant.

89. A vaccine composition comprising a therapeutically or prophylactically effective amount of a molecule as claimed in claim 67 and an adjuvant.

90. A vaccine composition comprising a therapeutically or prophylactically effective amount of an immunoglobulin as claimed in claim 73 and an adjuvant.

91. A vaccine composition comprising a therapeutically or prophylactically effective amount of an immunoglobulin as claimed in claim 74 and an adjuvant.

92. A method of treating or preventing cancer in a subject in need of such treatment or prevention, said method comprising administering to said subject a disease treating or preventing effective amount of a variant as claimed in claim 59 and an adjuvant.

93. A method of treating or preventing cancer in a subject in need of such treatment or prevention, said method comprising administering to said subject a disease treating or preventing effective amount of a molecule as claimed in claim 67 and an adjuvant.

94. A method of treating or preventing cancer in a subject in need of such treatment or prevention, said method comprising administering to said subject a disease treating or preventing effective amount of an immunoglobulin as claimed in claim 73 and an adjuvant.

95. A method of treating or preventing cancer in a subject in need of such treatment or prevention, said method comprising administering to said subject a disease treating or preventing effective amount of an immunoglobulin as claimed in claim 74 and an adjuvant.

96. A method of treating or preventing cancer as claimed in claim 91, wherein said cancer is selected from the group consisting of gastrointestinal cancer, breast cancer, small cell lung cancer, and medullary carcinoma of the thyroid.

97. A method of treating or preventing cancer as claimed in claim 92, wherein said cancer is selected from the group consisting of gastrointestinal cancer, breast cancer, small cell lung cancer, and medullary carcinoma of the thyroid.

98. A method of treating or preventing cancer as claimed in claim 93, wherein said cancer is selected from the group consisting of gastrointestinal cancer, breast cancer, small cell lung cancer, and medullary carcinoma of the thyroid.

99. A method of treating or preventing cancer as claimed in claim 94, wherein said cancer is selected from the group consisting of gastrointestinal cancer, breast cancer, small cell lung cancer, and medullary carcinoma of the thyroid.

100. A method of treating or preventing tumor metastasis in a subject in need of such treatment or prevention, said method comprising administering to said subject a disease treating or preventing effective amount of a variant as claimed in claim 59 and an adjuvant.

101. A method of treating or preventing tumor metastasis in a subject in need of such treatment or prevention, said method comprising administering to said subject a disease treating or preventing effective amount of a molecule as claimed in claim 67 and an adjuvant.

102. A method of treating or preventing tumor metastasis in a subject in need of such treatment or prevention, said method comprising administering to said subject a disease treating or preventing effective amount of an immunoglobulin as claimed in claim 73 and an adjuvant.

103. A method of treating or preventing tumor metastasis in a subject in need of such treatment or prevention, said method comprising administering to said subject a disease treating or preventing effective amount of an immunoglobulin as claimed in claim 74 and an adjuvant.

104. A method of eliciting an anti-idiotypic response to a tumor antigen in a subject in need of treatment or prevention of a disease condition associated with said tumor antigen, said method comprising administering to said subject a disease treating or preventing effective amount of a variant as claimed in claim 59 and an adjuvant.

105. A method of eliciting an anti-idiotypic response to a tumor antigen in a subject in need of treatment or prevention of a disease condition associated with said tumor antigen, said method comprising administering to said subject a disease treating or preventing effective amount of a molecule as claimed in claim 67 and an adjuvant.

106. A method of eliciting an anti-idiotypic response to a tumor antigen in a subject in need of treatment or prevention of a disease condition associated with said tumor antigen, said method comprising administering to said subject a disease treating or preventing effective amount of an immunoglobulin as claimed in claim 73 and an adjuvant.

107. A method of eliciting an anti-idiotypic response to a tumor antigen in a subject in need of treatment or prevention of a disease condition associated with said tumor antigen, said method comprising administering to said subject a disease treating or preventing effective amount of an immunoglobulin as claimed in claim 74 and an adjuvant.
